SEQUENCE LISTING (20 sequences)

SEQ ID NO: 1 - RNA, 20 nt, Homo sapiens
  source 1..20: mol_type = rRNA
cacgtctgatctgaggtcgc

SEQ ID NO: 2 - RNA, 20 nt, Homo sapiens
  source 1..20: mol_type = rRNA
atgctactggcaggatcaac

SEQ ID NO: 3 - RNA, 21 nt, Homo sapiens
  source 1..21: mol_type = rRNA
cgcacgagccgagtgatccac

SEQ ID NO: 4 - RNA, 18 nt, Homo sapiens
  source 1..18: mol_type = rRNA
ggtggtatggccgtagac

SEQ ID NO: 5 - RNA, 25 nt, Homo sapiens
  source 1..25: mol_type = other RNA
cactacagcccagaactcctggact

SEQ ID NO: 6 - RNA, 21 nt, Homo sapiens
  source 1..21: mol_type = tRNA
ctaccactgaaccacccatgc

SEQ ID NO: 7 - RNA, 30 nt, Homo sapiens
  source 1..30: mol_type = tRNA
ctgatgctctaccgactgagctatccgggc

SEQ ID NO: 8 - RNA, 21 nt, Homo sapiens
  source 1..21: mol_type = tRNA
tcaccactatactaacgagga

SEQ ID NO: 9 - RNA, 18 nt, Homo sapiens
  source 1..18: mol_type = tRNA
taaccactagaccaccag

SEQ ID NO: 10 - RNA, 28 nt, Homo sapiens
  source 1..28: mol_type = tRNA
gccgggaatcgaacccgggcctcccgcg

SEQ ID NO: 11 - RNA, 20 nt, Homo sapiens
  source 1..20: mol_type = tRNA
tcccgcgtggcaggcgagaa

SEQ ID NO: 12 - RNA, 21 nt, Homo sapiens
  source 1..21: mol_type = tRNA
cctcagattaaaagtctgatg

SEQ ID NO: 13 - RNA, 50 nt, Homo sapiens
  source 1..50: mol_type = other RNA
  variation 45..50
gtcgtatccagtgcagggtccgaggtattcgcactggatacgacnnnnnn

SEQ ID NO: 14 - RNA, 18 nt, Homo sapiens
  source 1..18: mol_type = tRNA
ccgcattggttcagtggt

SEQ ID NO: 15 - RNA, 25 nt, Homo sapiens
  source 1..25: mol_type = other RNA
gccccgtagcttatcagactgatgt

SEQ ID NO: 16 - RNA, 25 nt, Homo sapiens
  source 1..25: mol_type = tRNA
gctcggcattggtaattcagtggta

SEQ ID NO: 17 - RNA, 16 nt, Homo sapiens
  source 1..16: mol_type = other RNA
gtgcagggtccgaggt

SEQ ID NO: 18 - RNA, 38 nt, Homo sapiens
  source 1..38: mol_type = tRNA
gcgtctcacttatgcacagcgaacttgcatgggccggg

SEQ ID NO: 19 - RNA, 20 nt, Homo sapiens
  source 1..20: mol_type = tRNA
gcatgggtggttcagtggta

SEQ ID NO: 20 - RNA, 21 nt, Homo sapiens
  source 1..21: mol_type = other RNA
gtctcacttatgcacagcgaa

(19) United States
(12) Patent Application Publication
(10) Pub. No.: US 2024/0076748 A1
(43) Pub. Date: Mar. 7, 2024

(54) METHODS OF ENZYMATICALLY REPAIRING CLEAVED RNAS AND DETECTING THEREOF

(71) Applicants: INSTITUT PASTEUR DE MONTEVIDEO, Montevideo (UY); UNIVERSIDAD DE LA REPÚBLICA, Montevideo (UY); JOHNS HOPKINS UNIVERSITY, Baltimore, MD (US)

(72) Inventors: Juan Pablo Tosar, Montevideo (UY); Bruno Costa, Montevideo (UY); Alfonso Cayota, Montevideo (UY); Kenneth Witwer, Baltimore, MD (US)

(21) Appl. No.: 18/455,201

(22) Filed: Aug. 24, 2023

Related U.S. Application Data
(60) Provisional application No. 63/402,523, filed on Aug. 31, 2022.

(51) Int. Cl. C12Q 1/6886; C12N 9/00; C12N 9/12; C12N 15/10; C12Q 1/6806; C12Q 1/686

(52) U.S. Cl. CPC C12Q 1/6886; C12N 9/1205; C12N 9/93; C12N 15/101; C12Q 1/6806; C12Q 1/686; C12Y 207/01078; C12Y 605/01003

(57) ABSTRACT

The present disclosure relates to a method of enzymatically repairing RNAs that are nicked or at least partially cleaved. The method comprises providing a biological sample containing RNAs that are nicked or at least partially cleaved; purifying the nicked or at least partially cleaved RNAs contained in the biological sample, under a non-denaturing condition, to remove non-RNA components; and treating the purified RNAs with at least one of the following: (i) one or more enzymes that exhibit the activity of an RNA 3′ phosphatase or cyclic phosphatase and the activity of an RNA 5′ kinase, and an RNA ligase, or (ii) a 3′-5′ RNA ligase, thereby forming repaired RNAs from the nicked or at least partially cleaved RNA. The present disclosure also relates to a method of detecting RNAs by enzymatically repairing RNAs that are nicked or at least partially cleaved, and detecting the repaired RNAs.

### CROSS-REFERENCE TO RELATED APPLICATIONS

This application claims benefit of priority to U.S. Provisional Application No. 63/402,523, filed Aug. 31, 2022, which is herein incorporated by reference in its entirety.

This invention was made with government support under grant UG3/UH3CA241694 (Novel Separation Methods for exRNA Carriers: Extracellular Vesicles, Lipoprotein Particles, and Protein Aggregates) by the National Institutes of Health (National Cancer Institute and Office of the Director). The government has certain rights in the invention.

### SEQUENCE LISTING

The instant application contains a Sequence Listing which has been submitted in XML format and is hereby incorporated by reference in its entirety. Said XML copy, created on Aug. 21, 2023, is named IPDM9PCT_SL.xml and is 18,625 bytes in size.

### FIELD OF THE INVENTION

This invention relates to methods of enzymatically repairing nicked or partially cleaved RNAs and detecting RNAs.

### BACKGROUND

Nucleic acids-based disease monitoring (including diagnosis and prognosis) based on minimally invasive liquid biopsies has the potential to impact on the patients' survival rates by detecting diseases, relapse, or resistance to treatment in the patients at an early stage. However, this has been mostly studied in the context of extracellular cell-free DNA (cfDNA).

Extracellular RNAs (exRNA) can be more informative than cfDNAs because they also reflect gene expression changes across tissues and cell types. ExRNAs circulating in human bodily fluids can predict disease before the onset of clinical symptoms. For instance, exRNAs have been recently used successfully to predict preeclampsia in pregnant women at a pre-symptomatic stage (Moufarrej et al., 2022).

However, exRNAs are less studied and there still lacks fundamental knowledge on the types of exRNAs and their carriers in bodily fluids. When studying the exRNA world, a population composed of non-vesicular ribosomes and full-length tRNAs was uncovered (Tosar et al., 2020). Studies show that exRNA profiles may also contain from tRNA halves to full-length tRNAs (Nechooshtan et al., 2020). Moreover, stress can induce the formation of intracellular tRNA halves, potentially upregulating extracellular tRNA halves in EVs (Li et al., 2022; Tosar and Cayota, 2020). When encapsulated in extracellular vesicles (EVs), these fragments can be transferred to recipient cells (Gambaro et al., 2020), where they can trigger pattern recognition receptor-mediated signaling (Pawar et al., 2020; Xiao et al., 2020). However, most non-vesicular tRNA halves are generated directly in the extracellular space by endonucleolytic cleavage of extracellular full-length tRNAs (Nechooshtan et al., 2020; Sanadgol et al., 2022; Tosar et al., 2020). Extracellular ribosomes can induce dendritic cell activation in vitro in an exRNA-dependent manner (Tosar et al., 2020).

The studies on exRNAs have been more focused on the context of extracellular vesicles (EVs), which protect exRNAs from enzymatic degradation, because biofluids contain high quantities of enzymes that degrade RNA. Nonetheless, it has been known for years that the majority of exRNAs circulating in human blood plasma or in cell-conditioned medium are not associated with EVs (Arroyo et al., 2011; Turchinovich et al., 2011; Tosar et al., 2015). As a consequence of the strong ribonuclease activity that characterizes the extracellular space (Sorrentino, 2010), these non-vesicular exRNAs are expected to be rapidly degraded unless protected by RNA-binding proteins. It thus remains unknown on how these non-vesicular exRNAs resist degradation, diffuse to recipient cells, and trigger downstream effects, or even remain measurable and may serve as potential disease biomarkers. Explanations for some of these questions are provided in this invention, and the inventors have discovered a population of intrinsically stable non-vesicular extracellular RNAs, characterized by the presence of broken phosphodiester bonds in their structure (“nicked exRNAs”).

The problem is that nicked exRNAs cannot be identified by conventional or currently available sequencing and/or amplification methods. When reverse transcriptases are exposed to the broken phosphodiester bonds present in these RNAs, they fall off. As a result, these RNAs are either not represented in the sequencing or RT-qPCR assays, or they appear as RNA fragments, loosing important structural information and the physical association between 5′ and 3′ fragments.

There thus remains a need in the art to develop an effective method to more accurately characterize these exRNAs to enable their detection and/or subsequent applications (e.g., reverse transcription, amplification, analysis, hybridization, sequencing, etc.) that would have been undetectable in the biological sample due to the limits of the currently available RNA detection technology. This disclosure answers that need.

### SUMMARY OF THE INVENTION

One aspect of the invention relates to a method of enzymatically repairing RNAs that are nicked or at least partially cleaved. The method comprises providing a biological sample containing RNAs that are nicked or at least partially cleaved; purifying the nicked or at least partially cleaved RNAs contained in the biological sample, under a non-denaturing condition, to remove non-RNA components; and treating the purified RNAs with at least one of the following: (i) one or more enzymes that exhibit the activity of an RNA 3′ phosphatase or cyclic phosphatase and the activity of an RNA 5′ kinase, and an RNA ligase, or (ii) a 3′-5′ RNA ligase, thereby forming repaired RNAs from the nicked or at least partially cleaved RNAs.

Another aspect of the invention relates to a method for detecting RNAs from a biological sample. The method comprises providing a biological sample containing RNAs that are nicked or at least partially cleaved; purifying the nicked or at least partially cleaved RNAs contained in the biological sample, under a non-denaturing condition, to remove non-RNA components; treating the purified RNAs with at least one of the following: (i) one or more enzymes that exhibit the activity of an RNA 3′ phosphatase or cyclic phosphatase and the activity of an RNA 5′ kinase, and an RNA ligase, or (ii) a 3′-5′ RNA ligase, thereby repairing at least a portion of the nicked or partially cleaved RNAs; and detecting the repaired RNAs.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 depicts the results of the identification of stable non-vesicular RNAs. Northern blot of different rRNAs and tRNAs after incubating purified RNA from human cells, for different amounts of time in RPMI+10% FBS.

FIG. 2 shows that naked tRNA halves are extremely stable in human biofluids. A-C show the Northern blot of several noncoding transcripts after incubating purified RNA from human cells into 10% FBS or with recombinant human RNase 1 (A); into human urine or 10% serum (B); or in undiluted human serum (C). T=0 corresponds to RNAs added to serum and immediately placed on ice.

FIG. 3 shows the results of RNA decay varying concentrations of recombinant human RNase 1. A shows the Northern blot analysis using a probe complementary to the 5′ end of tRNA^{Gly}_{GCC} after the exposure of TRIzol-purified total U2-OS RNA (NT) to different concentrations of the r-RNase 1 at 37° C. for 60 minutes. B shows the Cloverleaf diagram of human tRNA^{Gly}_{GCC}−1 showing modified bases as described in the modomics database (http://genesilico.pl/modomics/) and predicted cleavage sites based on data presented in Example 1.

FIG. 4 shows the Northern blots corresponding to the assays shown in FIG. 2, A (A) and FIG. 2, B (B), but comparing the signals obtained when using probes complementary to the 5′ and 3′ halves of tRNA^{Gly}_{GCC}.

FIG. 5 shows that most tRNA halves identified by northern blot are nicked tRNAs. tRNA halves were generated in vitro by incubating RNA purified from human cells with recombinant human RNase 1 for 60 minutes. A shows the results of northern blot analysis where the RNase 1-treated RNA was incubated with T4 PNK, T4 PNK followed by T4 Rnl1, and T4 PNK followed by Rnl2, respectively. B is a schematic representation showing a summary of the nicked tRNA repair strategy. C depicts the 5′ and 3′ probes used in these assays, and a third probe targeting the anticodon loop of tRNA^{Gly}_{GCC} and tRNA^{Asp}_{GUC} (ACL). The assay described in A was then used. D shows the formation of nicked tRNAs was validated with the 5′, 3′ and ACL probes. Additional controls not included in A are also shown. NT: fragmented RNA not treated with the repair enzyme cocktail. Δ: heat. (−)PNK: T4 PNK lacking its phosphatase activity.

FIG. 6 shows the purification of RNAsel-treated RNA (with or without heating denaturation, Δ) by SPE, following manufacturer's instructions (1 volume of EtOH) or duplicating the volume of ethanol added to the binding buffer (2 volume of EtOH).

FIG. 7 shows that nicked tRNAs protect tRNA halves from degradation, are dissociated by phenol, and can be repaired by RtcB. A and B show that the RNA purification by the miRNeasy micro kit (A) or TRIzol (TRI, B) impaired enzymatic (PNK+Rnl1) repair of nicked tRNA (light gray arrows). The right panel in B shows the size of the repaired product estimated by the R_{f} method. C shows the purification of RNase1-treated RNA by SPE and re-exposure to RNase 1, with or without heating and then cooling the RNA. D shows the Northern blot of RNAsel-treated RNA purified by SPE, TRIzol, miRNeasy, or heated before the SPE purification, after separation in native gels. E shows a one-step enzymatic repair of nicked tRNA with RtCB from E. coli. The right panel shows the size of the repaired product estimated by the R_{f} method. In this and all subsequent figures, SPE was carried out with twice the recommended volume of EtOH.

FIG. 8 shows that non-vesicular tRNA halves circulating in serum and intracellular tRNA-derived fragments are predominantly nicked tRNAs. A shows that the purified RNA from arsenite-treated U2-OS cells was separated on a Superdex 75 column using an FPLC system. Inset: northern blot of intracellular RNAs showing the presence of tRNA halves in the input. A shows the selected fractions from this separation. B-C show that the selected fractions from (A) were analyzed by northern blot (B) or by stem-loop RT-qPCR (C). Fractions 2, 3 and 4 correspond to the elution volume of full-length tRNAs. C_{q} values were normalized to the fraction containing the highest signal. A-C bottom panels: the RNA was heated at 90° C. and then cooled down to room temperature before injection. D shows that cells were transfected with synthetic RNA 9^{GG/AA}, then lysed with SDS as described in (Tosar et al., 2018). The lysate was separated by SEC and fractions analyzed by SL-RT-qPCR using primers specific for the 9GG/AA oligonucleotide. E shows the separation by SEC of purified RNA from Proteinase K-treated ultracentrifugation supernatants of human serum. Selected eluted fractions were heated and analyzed by SL-RT-qPCR using primers specific for tRNA^{Gly}_{GCC} 5′ halves of 30 nt (solid line) and miR-21-5p (dotted line). The detection of tRNA halves in heated fractions corresponding to the elution volume of full-length tRNAs demonstrates the presence of nicked tRNAs in human serum.

FIG. 9 shows that the enzymatic repairing process allowed for an efficient reverse transcription and amplification (and hence, sequencing) of nicked tRNAs. A shows end-point RT-PCR and quantitative reverse-transcription PCR (RT-qPCR) of full-length tRNA^{Gly}_{GCC}, reverse-transcribed at 50° C. with a thermostable reverse transcriptase, using primers depicted in the diagram on the left. Input: total RNA from human cells. NT: RNase 1-treated total RNA (i.e., nicked tRNAs), purified under non-denaturing conditions, without enzymatic repair. PNK+Rnl1: RNase 1-treated RNA after enzymatic repair. Δ+PNK+RNl1: RNase 1-treated RNA heated before enzymatic repair. RT-qPCR results are expressed as fold change versus input. The diagram on the left shows the inability of reverse transcriptases (represented as a truck) to read pass the discontinuity that is characteristic of nicked or damaged RNAs. B is a schematic representation of the mechanism of an exemplary enzymatic repairing process.

### DETAILED DESCRIPTION OF THE INVENTION

Studying the nicked or at least partially cleaved RNAs remains a challenge because standard protocols for RNA extraction, northern blotting, and RNA sequencing induce their artefactual denaturation. In this disclosure, the inventors have developed an approach to enable reverse transcription, amplification, analysis, and/or sequencing of stable extracellular RNAs, without missing highly structured, nicked or partially cleaved exRNAs. The nicked or partially cleaved RNAs are purified under a non-denaturing condition (e.g., with a phenol-free method; without heating) and then enzymatically repaired to enable subsequent reverse transcriptions and/or various detections.

### Definition

Unless defined otherwise, technical and scientific terms used herein have the same meaning as commonly understood by one of ordinary skill in the art. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed. revised ed., J. Wiley & Sons (New York, NY 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present disclosure. Other features and advantages of the disclosure will become apparent from the following detailed description, taken in conjunction with the accompanying drawings, which illustrate, by way of example, various features of embodiments of this invention. Indeed, the present disclosure is in no way limited to the methods and materials described herein. For purposes of the present disclosure, certain terms are defined below.

“Treated” or “treatment” as used herein in the context of an assay means applying an effective amount of a substance under conditions that allow for the action of the substance. For example, “treating a sample with an enzyme” means applying a sufficient amount of an enzyme and under the appropriate conditions (buffers, temperature, etc.) to allow for an enzymatic reaction, as would be recognized by one of skill in the art.

A “nicked or partially cleaved RNA” or “nicked or at least partially cleaved RNA” generally refers to an RNA molecule having a discontinuity between adjacent nucleotides and/or having a portion removed from the parent, full-length RNA. For instance, a nicked or partially cleaved RNA may refer to an exRNA such as a tRNA having a cleaved phosphodiester bond in the anticodon loop. A nicked or partially cleaved RNA may also include a scenario where the RNA has a region (e.g., a loop or overhang or a portion thereof) removed from the parent, full-length RNA. A nicked or partially cleaved RNAs would include those artificially-generated RNA fragments, i.e., those generated by human interventions such as some experimental and/or treatment methods that would dissociate or completely cleave the nicked or partially cleaved RNAs into fragments. However, if an RNA fragment is not generated by human intervention, i.e., if different parts of the nicked or partially cleaved RNAs are already fragmented and separated in a biological sample that is used as an input, before the collection of said sample and before any human intervention such as experiment and/or treatment methods to biological sample, then these RNA fragments would typically not be considered as a nicked or partially cleaved RNA.

A “repaired RNA” refers to an RNA that is regenerated by ligating at least two portions or fragments (e.g., 5′ fragment and 3′ fragment) of a nicked or partially cleaved RNA, produced by cleaving the parent RNA, and reconstitutes the parent RNA's full-length or substantial full-length. For instance, for a nicked or partially cleaved tRNA, after the enzymatic repairing disclosed herein, the repaired RNA can refer to an RNA that is regenerated by ligating the 5′ half and the 3′ half of the nicked or partially cleaved tRNA, which are produced by cleaving the parent tRNA (or the nicked or partially cleaved tRNA), thereby reconstituting the parent RNA in full-length or substantial full-length. The repaired RNA can have the same length as the parent RNA, i.e., it is reconstituted to the parent RNA's full-length. Or the repaired RNA can have a length that is substantially the same as the parent RNA, i.e., it is reconstituted to the parent RNA's substantial full-length. By “substantial” full-length, the length of the repaired RNA may be at least 80%, at least 85%, at least 90%, at least 95%, at least 96%, at least 97%, at least 98%, at least 99%, or virtually 100% of the full length of the parent RNA. The repaired RNA can comprise a nucleotide sequence identical or substantially identical to the parent RNA. By “substantially identical,” the sequence of the repaired RNA may be at least 90%, at least 95%, at least 96%, at least 97%, at least 98%, at least 99%, or virtually 100% identical to sequences of the parent RNA.

### Extracellular RNAs

Extracellular (exRNAs) circulating in human bodily fluids can predict disease before the onset of clinical symptoms. Beyond translational applications as disease biomarkers in liquid biopsies (Heitzer et al., 2019), exRNAs are also involved in intercellular communication pathways between cells in different tissues (Thomou et al., 2017) and in host-pathogen interactions (Buck et al., 2014; Cai et al., 2018; Garcia-Silva et al., 2014).

One key aspect governing both exRNA functionality and utility as biomarkers is their stability against ubiquitous extracellular RNases (Tosar et al., 2021). This can be achieved by RNA encapsulation inside extracellular vesicles (EVs) such as exosomes and microvesicles (Skog et al., 2008; Valadi et al., 2007). However, despite the functional relevance (Mateescu et al., 2017) and biotechnological applications (O'Brien et al., 2020) of EV-encapsulated RNA, the majority of exRNAs in cell culture (Tosar et al., 2015; Turchinovich et al., 2011; Wei et al., 2017; Zhang et al., 2021) and in human plasma (Arroyo et al., 2011; Geekiyanage et al., 2020; Turchinovich et al., 2011; Vickers et al., 2011) are not transported as part of EV cargo, and are non-vesicular.

### Non-Vesicular RNAs

Cells can release ribosomes and full-length tRNAs into the extracellular space, where they can trigger immune cell activation. However, because non-vesicular exRNAs are prone to the action of extracellular ribonucleases, it has been considered unlikely that they could act as mediators of intercellular communication.

Contrary to common belief, the inventors have discovered that certain extracellular, non-vesicular RNAs are intrinsically stable against enzymatic degradation and show very high half-lives (from minutes to hours) when incubated in human biofluids at physiological condition (e.g., at 37° C.). These intrinsically stable non-vesicular RNAs circulate in human bodily fluids and are not associated with EVs. These intrinsically stable non-vesicular RNAs are very abundant inside most cells, but some RNAs can have tissue-specific expression. Cell damage or death may be the main source of non-vesicular RNAs in extracellular samples. Thus, intrinsically stable non-vesicular RNAs can be elevated in biofluids in situations where non-physiological quantities of cell damage or death are happening in the body (e.g., trauma, ischemia-reperfusion, cancer, autoimmunity, etc.).

Without being bound by theory, characteristics shared by the intrinsically stable non-vesicular RNAs include but are not limited to: a) they are highly structured and contain high % of nucleobases in double-stranded form, even for the ones that are single-stranded; b) they bear modified nucleotides; and c) they bear broken phosphodiester bonds (i.e., they are “nicked RNAs”) due to partial RNA cleavage by extracellular ribonucleases (RNases). This partial cleavage is not sufficient to break the structure of these molecules, which remain as dsRNAs stabilized by several internal base-pairing interactions.

tRNAs

Transfer RNA (tRNA) is a small RNA molecule that plays a key role in protein synthesis. Transfer RNA serves as a link (or adaptor) between the messenger RNA (mRNA) molecule and the growing chain of amino acids that make up a protein. tRNA typically has 70-100 (e.g., 76-90) nucleotides in length (in eukaryotes). tRNAs can be cleaved at the anticodon loop to produce “tRNA halves” that are 30-35 nucleotides in length in a process facilitated by the enzyme, Angiogenin, following induction of stress.

A tRNA fragment can be used to refer to functional short non-coding RNAs generated from a tRNA locus. tRNA fragments (tRFs) have lengths that range from 10 to 40 or more nucleotides. The term “tRNA locus” refers to the genomic region that includes a tRNA gene and gives rise to the tRNA transcript.

Transfer RNA-derived RNAs (tDRs) are among the most abundant nonvesicular small RNAs in cell culture (Tosar et al., 2015; Wei et al., 2017). Inside cells, tRNA cleavage and the consequent upregulation of specific tDRs is a conserved response to stress in all kingdoms of life (David et al., 1982; Li et al., 2008; Thompson et al., 2008). In humans, RNase A superfamily members, such as RNase 5 (Angiogenin), are responsible for stress-induced tRNA cleavage at the anticodon, generating stress-induced tRNA halves or tiRNAs (Fu et al., 2009; Yamasaki et al., 2009). tiRNAs can regulate gene expression at various levels, including global inhibition of translation initiation (Ivanov et al., 2011), by sequestering eIF4G (Lyons et al., 2021). Other, shorter tDRs can bind to mRNAs and regulate their translation (Kim et al., 2017) or silence genes by a miRNA-like, Argonaute-dependent mechanism (Kuscu et al., 2018).

Extracellular tDRs were first reported in EVs from murine immune cells (Nolte'T Hoen et al., 2012) but were later shown to be present mainly outside vesicles in mouse serum (Dhahbi et al., 2013; Zhang et al., 2014). In human cancer cell lines, tDRs could be detected in vesicular fractions but were overwhelmingly more abundant in EV-depleted ultracentrifugation supernatants (Tosar et al., 2015). While a heterogeneous population of tDRs can be detected inside cells, extracellular non-vesicular tDRs are mainly 5′ tRNA halves of 30 or 31 nucleotides, derived from tRNA^{Gly} and tRNA^{Glu}. These specific fragments are also ubiquitous in human biofluids (Srinivasan et al., 2019). The extracellular enrichment of these fragments may be due to their enhanced stability against degradation, because tRNA^{Gly}_{GCC} 5′ halves of 30-31 nt can form RNase-resistant homodimers in vitro (Tosar et al., 2018). However, the inventors have found that nicked tRNAs are probably the form in which tDRs are transported in biofluids. The high stability of nicked tRNAs against degradation therefore contributes to explain the abundance of extracellular non-vesicular tDRs.

### Methods of Enzymatically Repairing RNAs

One aspect of the invention relates to a method of enzymatically repairing RNAs that are nicked or at least partially cleaved. The method comprises providing a biological sample containing RNAs that are nicked or at least partially cleaved; purifying the nicked or at least partially cleaved RNAs contained in the biological sample, under a non-denaturing condition, to remove non-RNA components; and treating the purified RNAs with at least one of the following: (i) one or more enzymes that exhibit the activity of an RNA 3′ phosphatase or cyclic phosphatase and the activity of an RNA 5′ kinase, and an RNA ligase, or (ii) a 3′-5′ RNA ligase, thereby forming repaired RNAs from the nicked or at least partially cleaved RNAs.

The biological sample containing RNAs that are nicked or at least partially cleaved are purified to remove non-RNA components, such as proteins, lipids, salts, etc. that could interfere with downstream analysis. The purification is carried out under a non-denaturing condition.

In some embodiments, the non-denaturing condition comprises a solid phase extraction (SPE), chromatographic methods (such as size exclusion chromatography (SEC) or ion exchange chromatography), RNA precipitation (e.g., precipitation with a polar solvent, such as acetone or an alcohol (e.g., ethanol or isopropanol)), or a combination thereof. In one embodiment, the non-denaturing condition comprises SPE, such as a silica-based solid phase extraction columns.

The method may also contain one or more separation steps to extract one or more specific types of RNA, prior to the enzymatic treatment. In one embodiment, the method further comprises density gradient separation to isolate non-vesicular RNAs. In one embodiment, the method further comprises chromatographic methods to isolate non-vesicular RNAs. In one embodiment, the method further comprises native electrophoresis to fractionate RNAs based on their sizes.

The method may also contain one or more enrichment steps, prior to the enzymatic treatment. In one embodiment, the method further comprises gel-purifying to enrich one or more RNAs or fragments having 25-100 nucleotides.

In some embodiments, the purification of the biological sample and enzymatic treatment of the purified RNAs are carried out in the absence of a condition that results in denaturation of an RNA molecule. The condition that can result in denaturation of an RNA molecule can comprise heating, an adapter ligation, a chemical denaturant, or combinations thereof. In one embodiment, the condition that can result in denaturation of an RNA molecule comprises employing a chemical denaturant, such as phenol. In one embodiment, the condition that can result in denaturation of an RNA molecule comprises heating. The purification used herein avoids denaturing conditions that can create an artefact of missing certain highly structured, nicked or partially cleaved exRNAs and instead detecting an increased population of degraded/denatured RNAs that are in fact caused by the denaturing conditions.

In some embodiments, the method of enzymatically repairing nicked or at least partially cleaved RNAs is carried out in absence of a condition that results in denaturation of an RNA molecule. In one embodiment, the method is carried out in absence of a condition comprising heating, an adapter ligation, a chemical denaturant, or combinations thereof.

As discussed above, the nicked or partially cleaved RNA may be an RNA molecule having a discontinuity between adjacent nucleotides and/or having a portion removed from the parent, full-length RNA. In one embodiment, the nicked or partially cleaved RNA comprises a tRNA having a discontinuity between adjacent nucleotides (e.g., a cleaved phosphodiester bond) in the anticodon loop of the tRNA. In one embodiment, the nicked or partially cleaved RNA comprises an RNA having a region (e.g., a loop or overhang or a portion thereof) removed from the parent, full-length RNA.

The biological sample can contain various types of RNAs and various types of nicked or partially cleaved RNA. All the nicked or partially cleaved RNAs may be enzymatically repaired, e.g., simultaneously, by this method. Thus, by this method, after the enzymatic treatment disclosed herein, the final product would contain all RNAs originally contained in the biological samples, including those nicked or partially cleaved RNAs that have now been repaired.

In some embodiments, at least a portion of the nicked or partially cleaved RNAs are formed from extracellular RNAs. In some embodiments, at least a portion of the nicked or partially cleaved RNAs are formed from intracellular RNAs.

In some embodiments, at least a portion of the nicked or partially cleaved RNAs are formed from non-vesicular RNAs. In some embodiments, at least a portion of the nicked or partially cleaved RNAs are formed from vesicular RNAs.

In some embodiments, the nicked or at least partially cleaved RNAs comprise one or more RNAs selected from the group consisting of tRNA (e.g., nicked and full length), rRNA, YRNA, 7SL RNA, 7SK RNA, snRNA, snoRNA, vaultRNA, Alu RNA, transposable element-derived RNA, pri-microRNA, pre-microRNA, mRNA exons, mRNA introns, 5′ UTR, 3′ UTRs, and fragments and combinations thereof. These RNAs can be codified by either the nuclear genome or the genome of an organelle (e.g., mitochondria). These RNAs are examples of intrinsically stable non-vesicular RNAs. These RNAs are usually longer than 40 nucleotides in length, but sometimes appear in biofluid as relatively short RNA fragments due to the use of denaturing methods (sRNA-seq and/or northern blotting) applied to nicked exRNAs.

In one embodiment, the nicked or at least partially cleaved RNAs comprise tRNA, tRNA fragment (tRFs), tRNA-derived RNAs (tDRs), and/or tRNA half.

In one embodiment, at least a portion of the nicked or at least partially cleaved RNAs comprise single stranded tRNA half.

For the enzymatic repair treatment, one or more enzymes can be used. Without being bound by theory, the enzyme(s) used would perform the functions of dephosphorylating at 3′ end of the nicked or at least partially cleaved RNA (forming a 3′-OH), phosphorylating at 5′ end of the nicked or at least partially cleaved RNA (forming a 5′-phosphate), and ligating the 5′-phosphate RNA to the 3′-OH RNA. The enzymatic treatment can be carried out by one enzyme that has all these functions, two enzymes with the combination of both of which having all these functions, or three enzymes each having a different function and the combination of all of which having all these functions.

In some embodiments, the purified RNAs is treated with (i)(a) a polynucleotide kinase (PNK) and an RNA ligase. In one embodiment, the polynucleotide kinase is added together with the RNA ligase. In one embodiment, the polynucleotide kinase is added first, followed by the addition of the RNA ligase. In one embodiment, the polynucleotide kinase used is T4 PNK. In one embodiment, the RNA ligase is T4 RNA ligase 1.

In some embodiments, the purified RNAs is treated with (i)(b) an RNA 3′ phosphatase or an RNA 2′,3′ cyclic phosphatase, an RNA 5′ kinase, and an RNA ligase, added together, or in sequential order. In one embodiment, the RNA 3′ phosphatase or RNA 2′,3′ cyclic phosphatase, the RNA 5′ kinase, and the RNA ligase are all added together. In one embodiment, the RNA 3′ phosphatase or RNA 2′,3′ cyclic phosphatase, and the RNA 5′ kinase are added first, followed by the addition of the RNA ligase. In one embodiment, the RNA 3′ phosphatase or RNA 2′,3′ cyclic phosphatase is added first, followed by the RNA 5′ kinase and the RNA ligase. In one embodiment, the RNA 3′ phosphatase or RNA 2′,3′ cyclic phosphatase is added first, followed by the addition of the RNA 5′ kinase, and followed by the addition of the RNA ligase. In one embodiment, the RNA ligase is T4 RNA ligase 1.

In some embodiments, the purified RNAs is treated with (ii) a 3′-5′ RNA ligase. In one embodiment, the 3′-5′ RNA ligase is RtcB ligase.

In some embodiments, the treatment of the purified RNAs according to (i) (inclusive of (i)(a) and (i)(b)) or (ii) is carried out at least in part in the presence of a cofactor. In one embodiment, the cofactor is an adenosine triphosphate (ATP) or guanosine-5′-triphosphate (GTP).

The nicked or at least partially cleaved RNAs contained in the biological sample are repaired to reconstitute the parent RNA's full-length or substantial full-length. Having longer reads (and more complete reads of these nicked or at least partially cleaved RNAs) provides more important structural information and confers more discriminative power (especially in the case of tRNAs, that contain a high number of very similar isoacceptors and isodecoders that can have tissue-specific and cancer-specific expression).

In some embodiments, the repaired RNA comprises a nucleotide sequence identical or substantially identical to the parent RNA from which the nicked or at least partially cleaved RNA was formed, in full-length or in substantial full-length.

In one embodiment, the repaired RNA comprises a nucleotide sequence identical or substantially identical to the parent RNA from which the nicked or at least partially cleaved RNA was formed, in full-length.

In some embodiments, the repaired RNA comprises a nucleotide sequence identical or substantially identical to the parent RNA from which the nicked or at least partially cleaved RNA was formed, in substantial full-length. For instance, the length of the repaired RNA may be at least 80%, at least 85%, at least 90%, at least 95%, at least 96%, at least 97%, at least 98%, at least 99%, or virtually 100% of the full length of the parent RNA.

In one embodiment, the repaired RNA is 1-11 nucleotides shorter compared to its parent RNA from which the nicked or at least partially cleaved RNA was formed.

In one embodiment, the repaired RNA is 3-5 nucleotides shorter compared to its parent RNA from which the nicked or at least partially cleaved RNA was formed, losing a single-stranded overhang.

In one embodiment, the repaired RNA is 3-7 nucleotides shorter compared to its parent RNA from which the nicked or at least partially cleaved RNA was formed, losing an anticodon loop or a portion thereof.

The source of the biological sample may be a biofluid, a cell, a tissue, an organ, or any combination thereof.

In some embodiments, the biological sample is from a cell. In one embodiment, the biological sample is from a cancerous cell. In one embodiment, the nicked or at least partially cleaved RNAs are the precursors of stress-induced tRNA halves (tiRNAs).

In some embodiments, the biological sample is from an extracellular biofluid. For instance, the extracellular biofluid may be blood, blood serum, blood plasma, urine, lymph, saliva, synovia, milk, cerebrospinal fluid, or a combination thereof.

In some embodiments, the method comprises further purifying the enzyme-treated RNAs.

### Methods of Detecting RNAs

The method described herein can be used to detect RNAs such as extracellular RNAs in a biological sample, with a particular focus on providing important information on the highly structured RNAs, such as those nicked or partially cleaved RNAs, that are typically not identified by the conventional or currently available sequencing and/or amplification methods. The method can monitor gene expression changes in tissues by sequencing extracellular samples and to monitor disease (diagnosis, prognosis, relapse, response to treatment, etc.) based on the analysis of liquid biopsies.

Accordingly, another aspect of the invention relates to a method for detecting RNAs from a biological sample. The method comprises providing a biological sample containing RNAs that are nicked or at least partially cleaved; purifying the nicked or at least partially cleaved RNAs contained in the biological sample, under a non-denaturing condition, to remove non-RNA components; treating the purified RNAs with at least one of the following: (i) one or more enzymes that exhibit the activity of an RNA 3′ phosphatase or cyclic phosphatase and the activity of an RNA 5′ kinase, and an RNA ligase, or (ii) a 3′-5′ RNA ligase, thereby repairing at least a portion of the nicked or partially cleaved RNAs; and detecting the repaired RNAs.

All above descriptions and all embodiments discussed in the above aspect relating to the method of enzymatically repairing RNAs that are nicked or at least partially cleaved, including various aspects of the biological sample, nicked or at least partially cleaved RNAs, various RNA purification, separation, and/or enrichment methods, non-denaturing conditions, various enzyme treatment protocols, and the structure and sequences of the repaired RNA are applicable to this aspect of the invention relating to a method for detecting RNAs from a biological sample.

The method described herein can be applied to vesicular sample, non-vesicular sample, or total extracellular sample. It can be applied to patients suffering from various disease or conditions or to healthy donors. It can be applied to humans or to animals in veterinary medicine.

After the enzymatic repairing of the RNAs that are nicked or at least partially cleaved in the biological sample, the RNAs can be further reverse transcribed with a conventional reverse transcriptase, or a thermostable reverse transcriptase. In one embodiment, the RNAs can be further reverse transcribed with a thermostable reverse transcriptase. A typical thermostable reverse transcriptase is one that can work at high temperatures, to break the internal structure of the RNAs, and is more tolerant to modified bases. Exemplary thermostable reverse transcriptase suitable for applications herein are retroelement-derived reverse transcriptases such as TGIRT III and retrovirus-derived reverse transcriptases such as Superscript IV, among others.

After the enzymatic repairing of the RNAs that are nicked or at least partially cleaved in the biological sample, the RNAs can be detected by various detection methods known in the art.

In some embodiments, the method for detecting RNAs from a biological sample is carried out in absence of a condition that results in denaturation of an RNA molecule. In one embodiment, the method is carried out in absence of a condition comprising heating, a chemical denaturant, or combinations thereof.

Alternatively, in some embodiments, a condition that results in denaturation of an RNA molecule, such as heating, chemical denaturants, or combinations thereof, may be introduced, but is introduced after the enzymatic repair step(s) (i.e., the enzymatic treating step(s)) described herein.

In some embodiments, the detecting comprises sequencing, amplification, nucleic acid hybridization, or a combination thereof.

In one embodiment, the detecting comprises quantitative RT-PCR (RT-qPCR).

In one embodiment, the detecting comprises a form of high-throughput sequencing, which can be any form of a high-throughput sequencing including but not limited to sequencing by synthesis, nanopore sequencing, or any related technology that is available to obtain nucleic acid sequences.

In one embodiment, the biological samples are treated with RNase inhibitors before or after the purifying the nicked or at least partially cleaved RNAs contained in the biological sample.

### EXAMPLES

The following examples are for illustrative purposes only and are not intended to limit, in any way, the scope of the present invention. To the extent that specific materials are mentioned, it is merely for illustrative purpose and is not intended to limit the invention. One skilled in the art may develop equivalent methods or reactants without the exercise of inventive capacity and without departing from the scope of the invention.

### Example 1— Enzymatic Repair of Nicked tRNAs in Human Biological Samples

### Materials and Methods

### Cell Culture

U2-OS cells were cultured in DMEM (Gibco) with 4.5 g/L D-glucose and 110 mg/L sodium pyruvate, supplemented with 10% fetal bovine serum (FBS) (Gibco), without antibiotics.

### Biofluid Samples

Blood and urine samples were collected at the Pasteur Institute of Montevideo. Blood samples were obtained by venipuncture from a healthy donor in serum blood collection tubes and centrifuged at 2,500 rpm for 15 minutes to separate serum, which was stored at −20° C. until use.

20 mL of a human urine sample was retrieved within 1 hour of collection from a healthy donor in a sterile container. The sample was transferred to a 10 mL Falcon tube and centrifuged at 300×g and 4° C. for 10 minutes, followed by centrifugation at 2000×g and 4° C. for 20 minutes. Supernatant was then stored at −20° C. until use.

### RNA Purification by Solid Phase Extraction (SPE)

SPE was carried out using Monarch RNA Cleanup Kits (10 μg binding capacity columns, New England Biolabs, NEB) except for the purification of stress-induced tRNA-derived fragments from U2-OS cells, where the Total RNA Miniprep Kit (NEB) was used. Purification was carried out following manufacturer's instructions, but in several experiments, the volume of ethanol added to the binding buffer was doubled to avoid small RNA loss.

Generation of Stress-Induced tRNA Halves and Intracellular RNA Purification

For the generation of stress-induced tRNA halves (tiRNAs), 500 μM of freshly made sodium arsenite (Sigma-Aldrich) were added to U2-OS cells grown to 90% confluence in DMEM+10% FBS. After 2 hours at 37° C., the medium was removed and cells were washed with warm 1×PBS prior to RNA extraction using a Total RNA Miniprep Kit (NEB). Manufacturer's instructions were followed, except for two modifications: i) cell lysis was performed by adding 2 mL of lysis buffer directly to the cell monolayer, followed by a short (5-minute) incubation at room temperature; and ii) twice the indicated volume of ethanol was added to the RNA sample after elution from the RNA-binding column.

### RNA Decay Assays

For determination of RNA half-lives in biofluids, 1 μg of heated and refolded (1 minute at 90° C. followed by 30 minutes at room temperature) U2-OS total RNA was added to 50 μL undiluted human urine, undiluted human serum, diluted human serum, or FBS (both 10% in 1×PBS). Additionally, 1 μg of heated and refolded total RNA was incubated at 37° C. for variable amounts of time with recombinant human RNase 1 at 0.5 μg/mL (r-RNase 1; Bon Opus Bio). Reactions were stopped by the addition of SPE binding buffer and RNA cleanup.

### In Vitro RNA Digestion

For in vitro generation of nicked tRNAs and/or tDRs, 1 μg heated and refolded U2-OS total RNAs was mixed with 40 μL of r-RNase 1 (diluted in PBS at 0.0625 μg/mL) for 15, 30 or 60 minutes at 37° C.

### RNA Ligation Assays

For ligation assays involving T4 RNA ligases, 5 μL of in vitro-digested RNA (or RNA purified from CCM) were incubated for 1 h at 37° C. in a 10 μL reaction containing 20 U of RI, 1 mM ATP, 1×T4 PNK reaction buffer, and desired enzyme combinations. Enzymatic cocktails included: 10 U T4 RNA ligase 1 (Rnl1, NEB) or T4 RNA ligase 2 (Rnl2, NEB) and/or 10 U of T4 PNK (wild-type or 3′ phosphatase minus, NEB).

As a control, RNA was heat-denatured before enzymatic treatment (1 minute at 90° C. and immediately placed on ice).

For RtcB ligation, reaction mixtures (10 μL; 1 hour at 37° C.) contained 5 μL of in vitro-digested digested RNA (with or without previous heat denaturation), 20 U of RI, 1×RtcB ligase buffer, 1 mM GTP, 1 mM Mn²⁺, and 1 μM RtcB ligase from E. coli (NEB). The reactions were stopped by addition of 2×RNA Loading Dye and analyzed by northern blot.

Identification of Nonvesicular Nicked tRNAs in Biofluids

A 200 μL healthy donor human serum sample was thawed and centrifuged at 2,000×g and 4° C. for 10 minutes and diluted in 12 mL of PBS. EV depletion was performed by ultracentrifugation at 256,000×g and 4° C. for 1 hour in an Optima XPN ultracentrifuge (Beckman Coulter) with a SW 40 Ti rotor. The supernatant was then concentrated by ultrafiltration to 200 μL, using 10.000 MWCO Amicon Ultra-15 Centrifugal filters (Merck). Then, 440 μL of the RNA Binding Buffer included in RNA Cleanup kits (NEB) was added to the concentrated supernatant and treated with 20 μL Proteinase K (QIAgen) for 30 minutes at 37° C. Nucleic acids were then purified by SPE and eluted in 50 μL nuclease free H₂O (Invitrogen). Samples were analyzed by size-exclusion chromatography using an FPLC system.

### Size Exclusion Chromatography (FPLC)

Nonvesicular samples from human serum, or total RNA from U2-OS cells stressed with sodium arsenite (with or without heat denaturation and refolding) were diluted in 1×PBS (500 μL) and centrifuged at 10.000×g and 4° C. for 10 minutes prior to being injected into a Superdex 75 10/300 column (GE).

Size-exclusion chromatography (SEC) was performed at 0.5 mL/minute in 0.2 μm-filtered 1×PBS with an Äkta Pure FPLC system and 0.2 mL fractions were collected while monitoring the absorbance at 260 and 280 nm. Nucleic acids from selected fractions were ethanol precipitated (700 μL absolute anhydrous ethanol; 100 μL 3M NaAc, pH=5.2; 0.5 μL Glycogen Blue) overnight at −20° C. and centrifuged at 12.000×g and 4° C. for 15 minutes. The pellet was washed with 500 μL 75% ethanol, centrifuged at 12.000×g and 4° C. for 15 minutes, and resuspended in 10 μL of nuclease free water (Invitrogen) before northern blot or stem-loop RT-qPCR.

### Northern Blotting

Northern blot was performed with DIG-labelled DNA probes as described in Tosar (Tosar et al., 2020). Band intensities were obtained by densitometry using GelQuant 2.0 software.

For denaturing northern blots, 5 μL of RNA were mixed with 5 μL of loading buffer containing 95% formamide, 1 mM EDTA, 0.02% SDS, 0.02% bromophenol blue and 0.01% xylene cyanol, heated to 65° C. for 5 minutes and ran in 10×10 cm 10% polyacrylamide gels containing 7M urea in 1×Tris-borate EDTA (TBE, pH 8.4).

For non-denaturing northern blots, the RNA samples were mixed with 1 μL of 6× native loading buffer, and run in gels containing 1×Tris-borate (pH 8.3) and 10 mM MgCl₂ (TB+Mg²⁺) gels. Gels were run for 80 minutes in 0.5×TBE or 0.5×TB+Mg²⁺ running buffer at room temperature, stained with 1×SYBR gold (Invitrogen), and then transferred to positively charged nylon membranes (Roche) using a semi-dry Trans-Blot Turbo Transfer System (Bio-Rad) in 0.5×TBE at constant I=0.3 A for 30 minutes.

The membranes were UV cross-linked and hybridized for 16 hours at 42° C. with digoxigenin-labeled DNA probes in DIG Easy Hyb solution (Roche). After hybridization, membranes were washed for 5 minutes at room temperature with low stringency wash buffer (twice, 2×SSC/0.1% SDS), 5 minutes at 42° C. with high stringency wash buffer (1×SSC/0.1% SDS), blocked for 30 minutes at room temperature with 1×blocking solution (Roche) and probed for 30 minutes with an alkaline phosphatase-labeled anti-digoxigenin antibody (Roche). The membranes were washed twice with 1×TBS-T for 5 minutes, and then incubated in detection buffer (Roche). Signals were then visualized with CDP-Star, ready-to-use (Roche) and detected using an Amersham ImageQuant 800 imager (GE Healthcare/Cytiva).

### Probes (5′ to 3′):

rRNA 28S 5′:
(SEQ ID NO: 1)
CACGTCTGATCTGAGGTCGC
rRNA 18S 5′:
(SEQ ID NO: 2)
ATGCTACTGGCAGGATCAAC
rRNA 5.8S:
(SEQ ID NO: 3)
CGCACGAGCCGAGTGATCCAC
rRNA 5S 5′:
(SEQ ID NO: 4)
GGTGGTATGGCCGTAGAC
7SL:
(SEQ ID NO: 5)
CACTACAGCCCAGAACTCCTGGACT
tRNA^{Gly}_{GCC} 5′:
(SEQ ID NO: 6)
CTACCACTGAACCACCCATGC
tRNA^{Lys}_{UUU} 5′:
(SEQ ID NO: 7)
CTGATGCTCTACCGACTGAGCTATCCGGGC
tRNA^{ASP}_{GUC} 5′:
(SEQ ID NO: 8)
TCACCACTATACTAACGAGGA
tRNA^{Glu}_{GUC} 5′:
(SEQ ID NO: 9)
TAACCACTAGACCACCAG
tRNA^{Gly}_{GCC} 3′:
(SEQ ID NO: 10)
GCCGGGAATCGAACCCGGGCCTCCCGC
tRNA^{Gly}_{GCC}/^{ASP}_{GUC} ACL:
(SEQ ID NO: 11)
TCCCGCGTGGCAGGCGAGAA
tRNA^{Lys}_{UUU} ACL:
(SEQ ID NO: 12)
CCTCAGATTAAAAGTCTGATG

All oligonucleotides were obtained from Integrated DNA Technologies (IDT, USA), and labeled with DIG Oligonucleotide Tailing Kit, 2nd generation (Roche), following manufacturer's instructions.

### Stem-Loop RT-qPCR.

Stem-loop RT-qPCR was used to amplify and quantify small RNAs following protocols used in Tosar et al. (Tosar et al., 2015, 2018). Briefly, specific cDNA of tRNA^{Gly}_{GCC} 5′ halves and miR-21-5p was obtained with SuperScript II (ThermoScientific). Purified RNAs were heated at 90° C. for 1 minute and immediately placed on ice before reverse transcription. qPCR was performed using a QuantStudio 3 Real Time PCR System (ThermoScientific) with FastStart Universal SYBR Green Master (Rox; Roche). 2^{-Cq} values were obtained and normalized against the fraction containing the highest signal.

### Primers (5′ to 3′)

Stem-loop RT primer (“X” denotes assay-specific 3′ overhangs):

(SEQ ID NO: 13)
GTCGTATCCA GTGCAGGGTC CGAGGTATTC GCACTGGATA
CGACXXXXXX
tRNA^{Gly}_{GCC} (5′ half, 35 nt, 3′ overhang):
GGCAGG
tRNA^{Gly}_{GCC} (5′ half, 30 nt, 3′ overhang):
GCGAGA
miR-21-5p (3′ overhang):
GTCAAC
tRNA^{Gly}_{GCC} (5′ half, F-primer):
(SEQ ID NO: 14)
CCGCATTGGTTCAGTGGT
miR-21-5p (F-primer):
(SEQ ID NO: 15)
gccccgTAGCTTATCAGACTGATGT
9^{GG/AA} (F-primer):
(SEQ ID NO: 16)
gctcgGCATTGGTAATTCAGTGGTA
Universal reverse primer:
(SEQ ID NO: 17)
GTGCAGGGTCCGAGGT

All primers were obtained from Integrated DNA Technologies (IDT, USA). Lower case letters in these primers indicate the existence of added bases to increase the melting temperature.

RT-PCR for Full-Length tRNAs.

2 μL of 10 ng/μL U2-OS total RNA (input) or RNase 1-treated RNAs (starting from an equivalent amount of input RNA) were mixed with 1 μL of 2 μM gene-specific RT primer and 1 μL of 10 mM (each) dNTP mix in a reaction volume of 11 μL, incubated for 5 min at 65° C., and chilled on ice for at least 1 min. Annealed RNAs were mixed with 4 μL 5× SuperScript IV (SSIV, Thermo) buffer, 1 μL 0.1M DTT, 20 U RI and 20 U SSIV RT. The reaction was incubated for 10 minutes at 65° C. and then for 10 minutes at 80° C. The cDNA was diluted ½ prior to qPCR or 1/10 for endpoint PCR.

Gene-specific RT primer for tRNAGlyGCC1-4:

(SEQ ID NO: 18)
GCGTCTCACTTATGCACAGCGAACTTGCATGGGCCGGG
tRNAGlyGCC-1 (F-primer):
(SEQ ID NO: 19)
GCATGGGTGGTTCAGTGGTA
Universal tRNA reverse primer:
(SEQ ID NO: 20)
GTCTCACTTATGCACAGCGAA

### Results

Identification of Naked RNAs that are Stable in the Presence of Serum

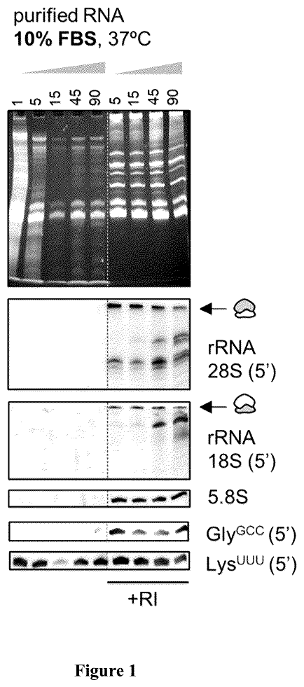

To search for intrinsically stable RNAs in extracellular samples, abundant cellular transcripts were screened to determine if they can resist degradation in serum-containing media in the absence of their protein counterparts (FIG. 1). As shown in FIG. 1, naked rRNAs were degraded in less than one minute, as illustrated by lack of northern blot signals in the absence of added RNase inhibitors (RI). In contrast, full-length tRNA^{Lys}_{UUU} was present at input levels after 1.5 hours, suggesting that this tRNA is not efficiently targeted by serum RNases. This was not observed for other tRNAs like tRNA^{Gly}_{GCC}, which, like rRNAs, was undetectable after one minute.

Naked full-length tRNA^{Lys}_{UUU} is intrinsically stable in biofluids

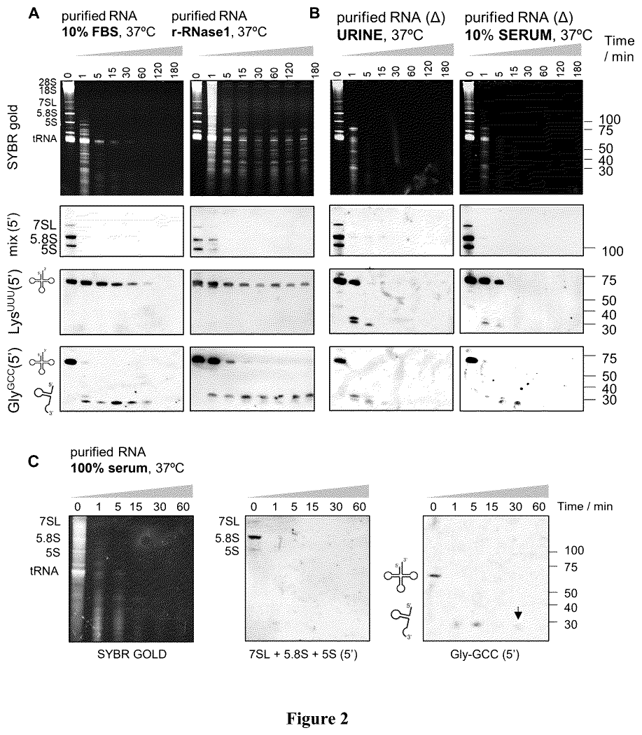

To precisely measure tRNA half-lives, the previous assay with better temporal resolution was repeated (FIG. 2, A). Strikingly, the half-life of tRNA^{Lys}_{UUU} (˜750 seconds) was 58-fold greater than that of tRNA^{Gly}_{GCC} (˜13 seconds) and >125-fold greater than that of rRNAs or the 7SL RNA (<6 seconds). The high stability of tRNA^{Lys}_{UUU} may not be explained by its association with serum-derived proteins, because the incubation with recombinant human RNase 1 (r-RNase1), representing the most common RNase in human blood, resulted in virtually identical results (FIG. 2, A). These results show that tRNA^{Lys}_{UUU} is intrinsically resistant to the action of RNase A family members.

These assays were then repeated in human biofluids including urine, diluted and undiluted serum (FIGS. 2, B and C). Surprisingly, the stability of full-length tRNA^{Lys}_{UUU} was always higher than the stability of any other tested RNA, irrespective of the sample type. Overall, there were profound differences in intrinsic extracellular stabilities not only between different RNA biotypes, but also within the same RNA biotype.

Glycine tRNA Halves Produced in Biofluids are Extremely Stable

Full-length tRNA^{Gly}_{GCC} was almost completely degraded in less than one minute in 10% FBS (FIG. 2, A) and in human biofluids (FIGS. 2, B and C). However, its cleavage resulted in the formation of 5′ halves that showed remarkably long half-lives, even in undiluted human sera.

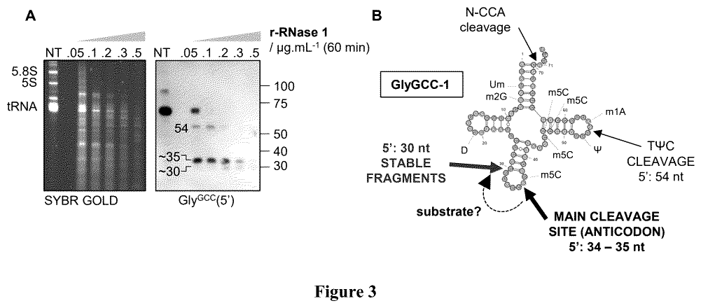

Closer inspection of the data in these figures shows the strong differences in the stability of fragments derived from the same parental tRNAs, but with slightly different lengths. tRNA^{Gly}_{GCC} was first cleaved at the anticodon loop, generating 34-35 nt 5′ halves that rapidly disappeared. These fragments were subsequently replaced by highly stable shorter fragments of approximately 30-31 nt, with a cleavage site at the start of the anticodon loop. An additional cleavage site in the TΨC loop (position 54) could be identified and exposed by using lower r-RNase1 concentrations (FIG. 3). The cleavage sites at position 30 and 34-35 may be independent, or the cleavage at position 34-35 may be a requisite for efficient cleavage at position 30.

Overall, tRNAs appeared to be more resistant to degradation than other longer noncoding RNAs, but differences in stability among tRNAs were also substantial (>50-fold). Also, naked tRNA^{Gly}_{GCC} 5′ halves of 30-31 nt could be accumulated at higher RNase concentrations or after longer incubations in biofluids.

Nicked tRNAs are a Source of 5′ and 3′ tRNA Halves

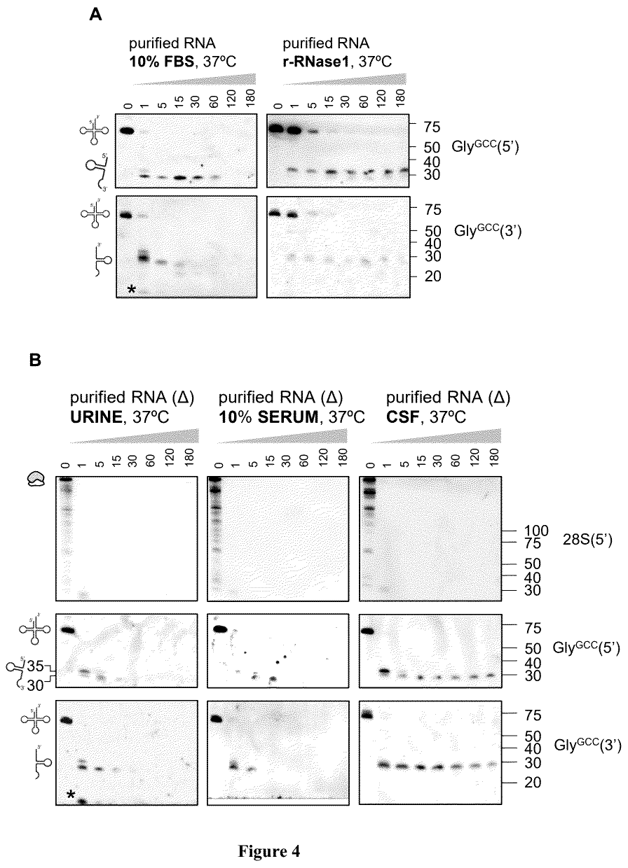

The relative stabilities of 5′ and 3′ tRNA^{Gly}_{GCC}-derived fragments were then compared, and the results are shown in FIG. 4. Surprisingly, 30-35 nt 3′ fragments were observed, and their rate of decay was comparable to their 5′ counterparts. In certain biofluids, such as FBS (FIG. 4, A) and urine (FIG. 4, B), 3′ tRNA-derived fragments <20 nt were observed at initial time points (1-5 minutes). However, these fragments presented a very short half-life, in sharp contrast with the 5′ and 3′ halves.

Without being bound by theory, tRNA^{Gly}_{GCC} 5′ and 3′ halves could remain physically associated with each other after RNase cleavage, representing a full-length tRNA bearing a cleaved phosphodiester bond at the anticodon loop (i.e., in the form of “nicked tRNAs”). This would explain the similar decay kinetics of each half among different biofluids. However, the introduction of any irreversible denaturation steps, such as those used in standard molecular biology approaches, would induce dissociation of nicked tRNAs into single-stranded tRNA halves. This can be further tested by new assays discussed herein capable of interrogating oligomeric RNA complexes under native conditions.

Nicked tRNAs are the natural substrates of T4 polynucleotide kinase (PNK) and T4 RNA ligase 1 (Rnl1) (Schwer et al., 2004). When Escherichia coli is infected by the T4 Phage, a bacterial anticodon nuclease called PrrC is activated and cleaves the host's tRNA^{Lys} in an attempt to prevent the translation of viral proteins (Kaufmann, 2000). This would result in a nicked tRNA bearing a 3′ cyclic phosphate (3′ cP) and a 5′-OH adjacent to the cleavage site. However, the phage evolved two enzymes capable of performing end-healing (PNK) and tRNA repair (Rnl1). These enzymes can be used to investigate the native structure of human tRNA halves in extracellular samples.

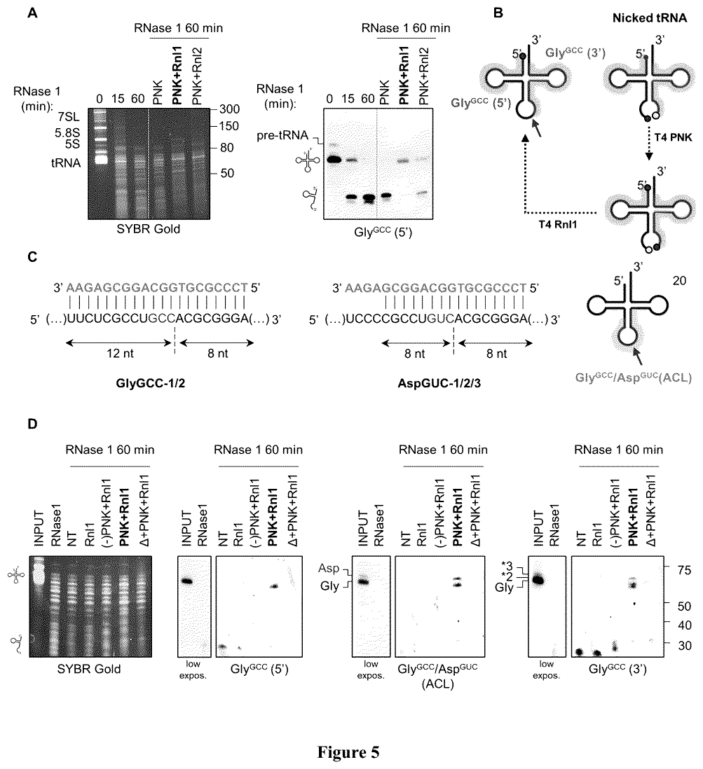

Total RNAs from cells were purified and incubated with r-RNase1 for 0, 15 or 60 minutes (FIG. 5, A). By 60 minutes, full-length tRNA^{Gly}_{GCC} was completely degraded and converted to a collection of tDRs (FIG. 5, A). RNase 1 degradation products were purified by silica-based solid phase extraction (SPE) columns and treated with either PNK alone, PNK followed by Rnl1, or PNK followed by Rnl2 (a dsRNA-specific ligase). Surprisingly, treatment with PNK and Rnl1 in tandem regenerated a single band of approximately the size of the cognate full-length tRNA (FIG. 5, A-B), and was more efficient than the ligation with Rnl2.

To demonstrate that the tRNA-sized re-ligated products are indeed repaired tRNAs, a third probe (termed ACL, for anticodon loop) bridging both sides of the anticodon of tRNA^{Gly}_{GCC} (FIG. 5, C) was designed, so that the Tm of its pairing with either the 5′ or the 3′ half was below the hybridization temperature of the assay (42° C.). Thus, the ACL probe would fail to detect 5′ or 3′ tDRs but should be able to hybridize with full-length or repaired tRNA^{Gly}_{GCC}. Due to sequence similarities among tRNAs, ACL would also recognize the anticodon loop of tRNA^{Asp}_{GUC}. But these tRNAs migrate slightly differently in denaturing urea gels, making this assay multiplex.

Strikingly, the treatment of RNase 1-treated RNA with PNK and Rnl1 regenerated tRNA-sized products observable with the 5′, the 3′, or the ACL probes (FIG. 5, D). In the case of ACL, two bands corresponding to the size of tRNA^{Gly}_{GCC} and tRNA^{Asp}_{GUC} were detected after the treatment with the enzymatic cocktail, in the context of an otherwise empty northern blot. Because the sequence patch recognized by the ACL probe was too short in the absence of enzymatic repair, this weighs against the possibility of a ligation in trans (an artefactual ligation product) between tRNA halves and other RNAs present in the sample. Furthermore, heating and then cooling the RNase-treated RNA before the addition of the enzymatic cocktail prevented the generation of a tRNA-sized band, demonstrating that the ligation occurred in cis (real nicked RNAs) under the assay conditions. Rnl1 alone or following incubation with a mutant version of T4 PNK lacking its 3′ phosphatase activity also failed to reconstitute a full-length tRNA.

In summary, nicked tRNAs were produced in vitro using different tRNAs as substrates. These nicked tRNAs were repaired enzymatically to regenerate an almost full-length tRNA, presumably lacking the 3′ NCCA overhang in in vitro settings (Akiyama et al., 2022). Additional bases can be trimmed after prolonged exposure to extracellular RNases, but these shorter forms would still be reparable using the assay discussed herein.

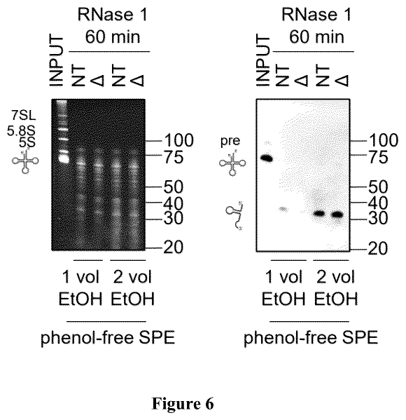

Nicked tRNAs are Irreversibly Melted by Popular RNA Extraction Methods

Whereas heating RNase-treated RNA before tandem incubation with PNK and Rnl1 prevented the formation of a tRNA-sized band, this also affected the detection of monomeric 5′ and 3′ tRNA halves (FIG. 5, D). It was also observed that heating alone was sufficient to reduce the intensity of the northern blot band corresponding to the tRNA halves (FIG. 6).

The size cut-off of SPE RNA cleanup columns can be reduced by doubling the amount of ethanol added to the sample. When two volumes of ethanol were used, the amount of eluted tRNA halves was increased, while the effect of heat was lost. This indicates that nicked tRNAs (>70 nt) can bind to the SPE columns when one volume of ethanol is used, but monomeric 30-35 nt tRNA halves are lost in the flow-through. In contrast, doubling the ethanol allows a highly efficient capture of both nicked tRNAs and heat-induced monomeric tRNA halves. The enzymatic repair assays were then repeated under conditions desirable for small RNAs (2×EtOH) where the monomeric tRNA halves were still detectable in the control (i.e., heat) reaction (FIG. 6).

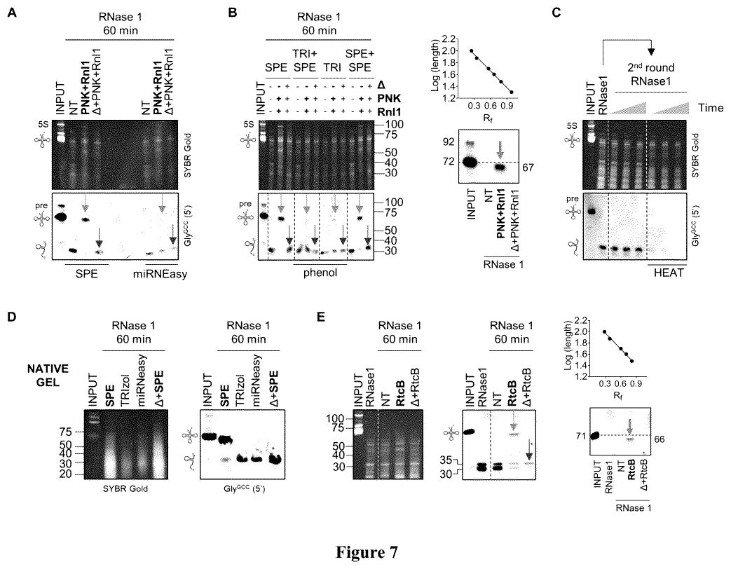

Given that heating irreversibly affected nicked tRNAs, the effects of various commonly used RNA extraction methods were studied, many of which include agents (e.g., phenol) known to disrupt base-pairing interactions. A recent study of different RNA purifications methods for liquid biopsies found that the miRNeasy kit (Qiagen) recovered a broad spectrum of exRNAs associated with different carrier subclasses (Srinivasan et al., 2019). However, when RNase1-treated RNA was purified with this kit following the manufacturer's instructions, tRNA halves were recovered at acceptable yields but were no longer amenable to enzymatic repair (FIG. 7, A). Similar results were obtained when comparing phenol-free SPE-based purification vs. TRIzol (FIG. 7, B, left). Interestingly, adding a second SPE-based purification round recovered reparable tRNA halves with a yield close to 100%. Thus, guanidine salts included in the SPE binding buffer would not affect nicked tRNAs, while heating and phenol would induce their irreversible separation. Also, the length of the repaired tRNA (estimated by the R_{f} method, FIG. 7, B, right) was slightly shorter (4-5 nt) than that of the parental full-length tRNA.

Nicked tRNAs are Resistant to RNase 1 Cleavage In Vitro

Having observed that nicked tRNAs can be purified by SPE, nicked tRNAs were generated and purified, and were treated again with r-RNase 1 (FIG. 7, C). Nicked tRNAs were not degraded by this treatment (up to 30 minutes at 37° C.). However, heating and then cooling the nicked tRNAs before exposure to r-RNase1 induced complete degradation of the tRNA halves, now in their single-stranded form.

In conclusion, nicked tRNAs are stable reservoirs of tRNA halves, which are degradation-prone once being dissociated from their 3′ counterparts.

In Vitro Generated tRNA Halves are Predominantly Nicked tRNAs

A comparison of the intensities of enzymatically repaired (light gray arrows) and untreated (NT) tRNA^{Gly}_{GCC} 5′ halves in RNase1-treated samples purified under optimized conditions (FIG. 7, A-B) suggests that most tDRs were in fact nicked tRNAs.

A northern blot assay was also conducted after running the RNAs on a native (TB-Mg²⁺) polyacrylamide gel to further confirm this conclusion (FIG. 7, D). Under native conditions, the electrophoretic behavior of RNase1-treated tRNA^{Gly}_{GCC} was almost identical to that of untreated full-length tRNAs. Only <10% of the total signal in the treated lane corresponded to bona-fide tDRs. This confirms that the rapid disappearance of the full-length tRNA band observed in FIG. 2, A, was in fact an artefact caused by denaturing conditions, with >90% of tRNAs remaining as nicked tRNAs after 1 hour of enzymatic digestion. Interestingly, the migration of nicked tRNAs in native gels was slightly faster than that of unnicked full-length tRNAs, consistent with the irreversible loss of the NCCA 3′ overhang inferred from previous assays (FIG. 7, B).

Native northern blots also confirmed that heating or standard RNA purification methods such as TRIzol or miRNeasy, unlike phenol-free RNA cleanup columns (SPE), induced the irreversible dissociation of nicked tRNAs (FIG. 7, D).

The 3′-5′ RNA Ligase RtcB can Also Ligate Nicked tRNAs

RtcB is a ligase involved in tRNA splicing and RNA repair in all domains of life (Englert et al., 2011; Popow et al., 2011; Tanaka and Shuman, 2011). Unlike the 5′-3′ T4 Rnl1, RtcB seals broken RNAs with 2′,3′ cyclic phosphate (2,3-cP) and 5′-OH ends in the presence of GTP and Mn²⁺ (Chakravarty et al., 2012). This enzyme actually catalyzes a two-step process, where 2,3-cP is hydrolyzed to a 3′-monophosphate and subsequently ligated to the 5′-OH (Tanaka and Shuman, 2011).

FIG. 7, E (left), shows that a one-step enzymatic repair of nicked tRNA with RtCB from E. coli was sufficient to repair RNase1-treated RNAs without prior end-healing by T4 PNK. Indeed, RtcB regenerated a tRNA-sized band when incubated with RNase1-treated RNAs, and generation of this ligation product was inhibited by heating (FIG. 7, E, left). The repaired tRNA-sized band was 4 or 5 nucleotides shorter than the parental full-length tRNA (FIG. 7, E, right).

Nicked tRNAs as a Source of Intracellular Stress-Induced tRNA Halves

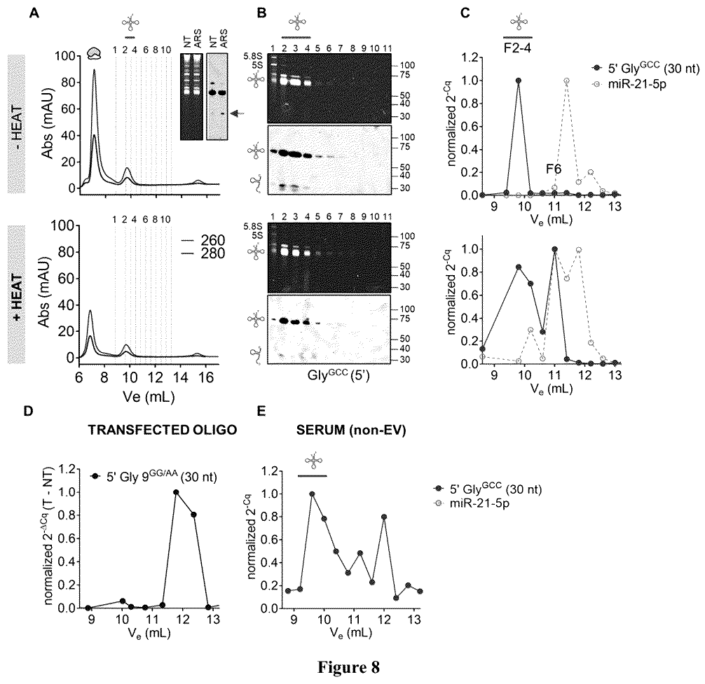

The enzymatic repair assays may not be as effective to detect the existence of nicked tRNAs inside cells when a large excess of full-length tRNAs are present in intracellular samples. A different strategy based on intracellular RNA fractionation under nondenaturing conditions by size-exclusion chromatography (SEC) (FIG. 8, A-C) was used herein, to separate stress-induced tRNA halves (tiRNAs) present inside cells (which are single-stranded fragments, even if complexed with proteins) from their parental tRNAs.

U2-OS cells were exposed to 500 μM sodium arsenite for two hours based on methods described in Yamasaki et al. (Yamasaki et al., 2009) and the presence of stress-induced tRNA halves was verified by northern blot (FIG. 8, A). Stressed cells were then lysed by phenol-free methods, and intracellular RNA was purified by SPE and separated by SEC using an FPLC system. As a control, the RNA was heat-denatured, cooled down to room temperature and injected in a parallel assay.

A tRNA peak was consistently eluted at Vₑ=9.80 mL, evidenced by the registered absorbance at 260 nm (FIG. 8, A) and northern blot (FIG. 8, B). Surprisingly, the northern blot bands consistent with 5′ tRNA halves (of 35 and 30 nt) were observed only in the fractions corresponding to the tRNA peak (FIG. 8, B). A more sensitive stem-loop RT-qPCR assay according to the method described in Tosar et al. (Tosar et al., 2015) was used to identify the tRNA halves in the other fractions. While miR-21-5p eluted at Vₑ=11.4 mL, tRNA^{Gly}_{GCC} 5′ halves of 30 nt were almost undetectable except at Vₑ=9.80 mL, where most full-length tRNAs elute (FIG. 8, C). Heating the RNA before injection decreased the northern blot signal in the full-length tRNA peak (FIG. 8, B) while shifting the main SL-RT-qPCR peak of tRNA^{Gly} 5′ halves to higher elution volumes. Most tRNA-derived fragments of 30 nt co-eluted with miR-21-5p in heated samples.

Dimerization of tRNA^{Gly}_{GCC} 5′ halves could also explain their elution at Vₑ=9.80 mL in these chromatographic columns (Tosar et al., 2018). However, the transfection of cells with 30 nt tRNA^{Gly}_{GCC} 9 GG/AA mutants, which can dimerize in vitro (Tosar et al., 2018), showed a chromatographic elution consistent with their presence in monomeric form (FIG. 8, D). This suggests that either intracellular conditions did not favor homodimer formation, or that dimers dissociated during cell lysis.

In sum, dimerization would not explain the elution of intracellular tRNA halves in chromatographic peaks corresponding to RNAs with twice their predicted size. The results thus suggest that at least some intracellular tiRNAs were predominantly present in the form of nicked tRNAs. Alternatively, intracellular tiRNAs might be degraded upon cell lysis, leaving only the more stable nicked tRNAs.

Nonvesicular Nicked tRNAs Circulate in Human Biofluids

Having validated a method capable of separating nicked tRNAs from single-stranded tRNA halves, this method was implemented to further address the question of whether nicked tRNAs circulate in human biofluids.

200 μL human serum was diluted in PBS, and EVs were pelleted by ultracentrifugation. RNA was then isolated from Proteinase K-treated supernatants and fractionated by SEC. Surprisingly, 30 nt tRNA^{Gly}_{GCC} 5′ halves could be amplified by SL-RT-qPCR (FIG. 8, E), mostly in the size range corresponding to full-length tRNAs, which were expected to be absent in these samples (FIG. 2, B). These results demonstrate that nonvesicular nicked tRNAs are present in human sera.

Nicked tRNAs are Reverse-Transcribed Inefficiently Unless Repaired.

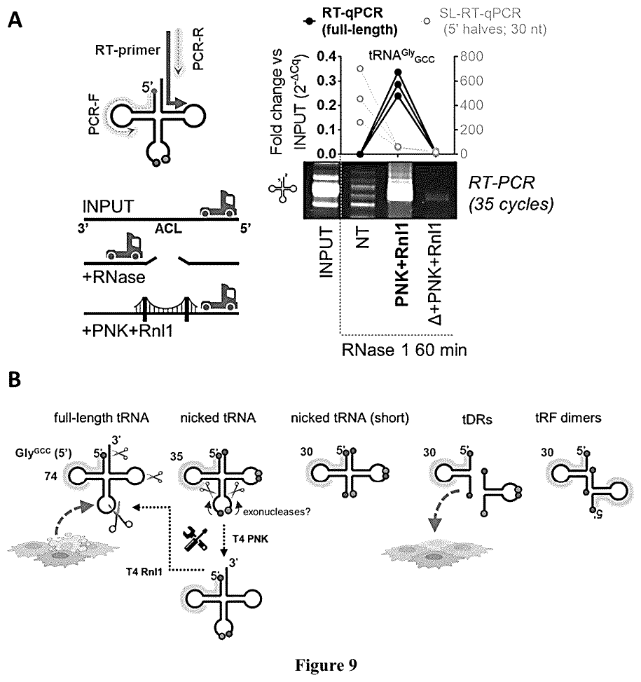

Broken phosphodiester bonds could act as roadblocks inhibiting reverse transcription (RT) of nicked tRNAs in their native state, preventing the analysis of nicked tRNAs by RT-PCR or sequencing. This effect was evaluated using a thermostable retroviral reverse transcriptase primed by a gene-specific RT primer aligning to the 3′ end of tRNA^{Gly}_{GCC} (FIG. 9A). Forward and reverse PCR primers were placed closed to the 5′ and 3′ ends of tRNA^{Gly}_{GCC}, respectively. Surprisingly, RNase-1-treated RNA was not amplified unless enzymatic repair (T4 PNK+T4 Rnl1) was performed before RT. Consistent with northern blot results as shown in FIGS. 7A, 7B, and 7E, heating the samples before the enzymatic treatment steps also inhibited RT-PCR amplification (see FIG. 9A). Conversely, 5′ tRNA^{Gly}_{GCC} halves of 30 nt increased 200 to 700-fold in RNase1-treated samples versus input, illustrating the limitations of small RNA expression analysis in samples containing nicked forms of their parental RNAs. FIG. 9A shows the inability of reverse transcriptases (represented as a truck) to read pass the discontinuity that is characteristic of nicked or damaged RNAs, indicating that the enzymatic repairing process as disclosed herein is needed for an efficient reverse transcription and amplification (and hence, sequencing) of nicked tRNAs.

### Discussion

It was widely believed that all RNAs are intrinsically unstable and cannot circulate in extracellular samples unless in the context of RNPs, lipoproteins and/or EVs. Although this holds true for rRNA-derived fragments (FIG. 1) and nonvesicular RNU2-derived small RNAs (Tosar et al., 2022), full-length or nicked tRNAs showed surprisingly long half-lives in human biofluids, even when incubated in their naked forms. While these RNAs might also be present in RNP complexes in the extracellular space, the results herein demonstrate that protein complexation is not a prerequisite for remarkable extracellular stability.

Importantly, the differences in extracellular stabilities were observed among tRNAs. At one extreme, the full-length tRNA^{Lys}_{UUU} was processed at slower rates than any other tested RNA, irrespective of the sample type. This was in sharp contrast with, for example, the rapidly processed full-length tRNA^{Gly}_{GCC}. One explanation is that post-transcriptionally modified bases are at least partially responsible for these different behaviors. Based on the modomics database, tRNA^{Gly}_{GCC} does not contain modified bases in the anticodon loop, except for m5C at position 37. In contrast, at least in yeast, tRNA^{Lys}_{UUU} contains mcm5s2U and t6A at positions 34 and 37, respectively. Although these modifications have been shown to facilitate cleavage by bacterial and yeast anticodon ribonucleases (Bacusmo et al., 2018; Lentini et al., 2018), mammalian RNases are often inhibited by modified bases present in the anticodon (Lyons et al., 2018).

The difference in the stability between tRNAs is less pronounced when considering that nicked tRNAs, and not tDRs, are the stable degradation intermediates dictating the abundance of nonvesicular glycine tRNA halves. Although the full-length tRNA^{Gly}_{GCC} was efficiently cleaved at several positions by extracellular RNases in human biofluids, the result of these cleavage events is a molecule that probably still resembles a tRNA, even if it bears some broken phosphodiester bonds. Thus, although tRNA^{Lys}_{UUU} and tRNA^{Gly}_{GCC} show completely different behaviors when analyzed by northern blot after being exposed to RNases, this difference could have been exaggerated because the standard northern blotting forced nicked tRNAs to denature. This would be also the case when purifying RNA using phenol and when RNA is heated at any stage in a protocol (e.g., RNA-seq). The important conclusion is the true, native form of RNA is not always being apprehended because most available analytical techniques contain denaturation steps at some point.

The case of tRNA^{Lys}_{UUU} is also interesting. Although it is highly resistant to degradation, once being cleaved by r-RNase 1 or by FBS-derived RNase A, it did not survive as a nicked tRNA (FIG. 2, A). This implicates extracellular RNases in the degradation of tDRs (Li et al., 2022). In contrast, tRNA^{Gly}_{GCC} was sensitive to initial cleavage events, but the nicked tRNAs produced therefrom were intrinsically stable. Differential stabilities among distinct tRNA sequences could be considered a new example of non-canonical “moonlighting” functions of tRNAs, what has been related to the diversity of the tRNA isodecoder pool (Avcilar-Kucukgoze and Kashina, 2020).

Nicked tRNAs were not considered as stable degradation intermediates in the literature (Chen et al., 2021). Stress-induced tRNA cleavage at the anticodon loop is thought to be an irreversible process, whereas reversibility of stress-induced nicked tRNA formation in cellulo arises as an exciting possibility. In the clinical setting, double-stranded RNAs usually require encapsulation in lipid nanoparticles or GalNAc conjugation for efficient uptake, but single-stranded oligonucleotides are spontaneously endocytosed (Levin, 2019). Thus, the mechanism of the enzymatic repairing process disclosed herein where the nonvesicular nicked tRNAs are carriers of tRNA halves that can transfer information to recipient cells is at least feasible (FIG. 9B).

Disclosed herein is a new analytical technique that can be used to analyze stable nonvesicular RNAs circulating in biofluids. Although this example focused mostly on tRNAs, close inspection of gels stained with SYBR gold shows additional bands that could be restored by combined treatment with PNK and Rnl1 and that were lost in heated samples (FIG. 7, B). This strongly suggests that the nonvesicular RNAome is much more complex than previously thought, in accordance with recent findings (Tosar et al., 2020). Many stable nonvesicular RNAs are single-stranded molecules tightly bound and protected by extracellular RNA-binding proteins (Arroyo et al., 2011; Tosar et al., 2022; Turchinovich et al., 2011). However, other resilient RNAs circulating in biofluids could belong to a “new” category of intrinsically-stable extracellular RNAs (Tosar, 2021). These molecules can be both highly structured and nicked.

There are sequencing methods that can efficiently handle highly structured RNAs (Behrens et al., 2021; Qin et al., 2016). However, nicked RNAs are more challenging because they contain roadblocks of reverse transcriptases (i.e., the broken phosphodiester bonds; FIG. 9A) and because nicked RNAs are dissociated by phenol, heat, or other denaturing agents. The enzymatic repair protocol disclosed herein therefore increases the number and types of RNA molecules that can be analyzed and used as disease biomarkers.

In this example, protein-free RNAs were incubated in human biofluids, and their decay kinetics were measured by northern blot. Certain specific naked tRNAs were found to be intrinsically stable. The half-lives of several naked RNAs were measured. Although rapid clearance was the norm, specific full-length tRNAs decayed comparatively slowly in biofluids. Furthermore, the stability of 5′ tRNA halves generated from the endonucleolytic cleavage of unstable tRNAs was extremely high. However, these ultra-stable tRNA-derived fragments (tDRs) are not present as real fragments in most extracellular samples. On the contrary, they circulate mainly as full-length tRNAs containing a few broken phosphodiester bonds. Also surprisingly, these ultra-stable RNAs were not single-stranded.

Studying these nicked tRNAs remains a challenge because standard protocols for RNA extraction, northern blotting and RNA-seq induce their artefactual denaturation, even though their presence is supported by native gels. Different strategies of enzymatic repair and electrophoretic or chromatographic separation under native conditions were developed and employed in this example. A two-step method was employed based on enzymatic repair with T4 PNK and Rnl1 (or a one-step ligation with recombinant RtcB) after phenol-free RNA purification. Heating the samples before the enzymatic treatment abrogated nicked tRNA repair. Nicked tRNAs were separated from tDRs by chromatographic methods under native conditions. These protocols were used to identify nicked tRNAs inside stressed cells and in vesicle-depleted human biofluids.

The results also show that these RNAs natively exist as full-length tRNAs containing broken phosphodiester bonds. This is surprising and important, because commonly used RNA purification methods, sequencing, and northern blotting do not detect these nicked or partially cleaved forms, forcing them to melt into single-stranded tRNA halves. In addition, FIG. 9 shows that the enzymatic protocol described herein is needed for efficient reverse transcription and amplification (and hence, sequencing) of nicked tRNAs. As shown in FIG. 9A, if nicked RNAs are purified under native conditions, without the enzymatic repair steps, they cannot be efficiently reverse-transcribed (and therefore amplified and sequenced). This same conclusion would apply to the nanopore-based direct RNA sequencing.

The method shown in this example is useful to uncover a hidden layer of the extracellular RNAome composed of intrinsically stable, nick-containing, highly structured RNAs.

All documents and references cited herein, including but are not limited to, journal articles or abstracts, published or corresponding U.S. or international patent applications, issued U.S. or foreign patents, or any other documents, are each incorporated herein by reference in their entirety, including all data, tables, figures, and text presented in the cited documents and references.

### REFERENCES

Akiyama, Y., Lyons, S. M., Fay, M. M., Tomioka, Y., Abe, T., Anderson, P. J., and Ivanov, P. (2022). Selective Cleavage at CCA Ends and Anticodon Loops of tRNAs by Stress-Induced RNases. Front. Mol. Biosci. 9.
Arroyo, J. D., Chevillet, J. R., Kroh, E. M., Ruf, I. K., Pritchard, C. C., Gibson, D. F., Mitchell, P. S., Bennett, C. F., Pogosova-Agadjanyan, E. L., Stirewalt, D. L., et al. (2011). Argonaute2 complexes carry a population of circulating microRNAs independent of vesicles in human plasma. Proc. Natl. Acad. Sci. U.S.A 108, 5003-5008.
Avcilar-Kucukgoze, I., and Kashina, A. (2020). Hijacking tRNAs From Translation: Regulatory Functions of tRNAs in Mammalian Cell Physiology. Front. Mol. Biosci. 0, 388.
Bacusmo, J. M., Orsini, S. S., Hu, J., DeMott, M., Thiaville, P. C., Elfarash, A., Paulines, M. J., Rojas-Benitez, D., Meineke, B., Deutsch, C., et al. (2018). The t6A modification acts as a positive determinant for the anticodon nuclease PrrC, and is distinctively nonessential in Streptococcus mutans. RNA Biol. 15, 508-517.
Behrens, A., Rodschinka, G., and Nedialkova, D. D. (2021). High-resolution quantitative profiling of tRNA abundance and modification status in eukaryotes by mim-tRNAseq. Mol. Cell 81, 1802-1815.e7.
Buck, A. H., Coakley, G., Simbari, F., McSorley, H. J., Quintana, J. F., Le Bihan, T., Kumar, S., Abreu-Goodger, C., Lear, M., Harcus, Y., et al. (2014). Exosomes secreted by nematode parasites transfer small RNAs to mammalian cells and modulate innate immunity. Nat. Commun. 5, 5488.
Cai, Q., Qiao, L., Wang, M., He, B., Lin, F.-M., Palmquist, J., Huang, S.-D., and Jin, H. (2018). Plants send small RNAs in extracellular vesicles to fungal pathogen to silence virulence genes. Science 360, 1126-1129.
Chakravarty, A. K., Subbotin, R., Chait, B. T., and Shuman, S. (2012). RNA ligase RtcB splices 3′-phosphate and 5′-OH ends via covalent RtcB-(histidinyl)-GMP and polynucleotide-(3′)pp(5′)G intermediates. Proc. Natl. Acad. Sci. U.S.A 109.
Chen, Q., Zhang, X., Shi, J., Yan, M., and Zhou, T. (2021). Origins and evolving functionalities of tRNA-derived small RNAs. Trends Biochem. Sci. 46, 790-804.
David, M., Borasio, G. D., and Kaufmann, G. (1982). Bacteriophage T4-induced anticodon-loop nuclease detected in a host strain restrictive to RNA ligase mutants. Proc. Natl. Acad. Sci. 79, 7097-7101.
Dhahbi, J. M., Spindler, S. R., Atamna, H., Yamakawa, A., Boffelli, D., Mote, P., and Martin, D. I. K. (2013). 5′ tRNA halves are present as abundant complexes in serum, concentrated in blood cells, and modulated by aging and calorie restriction. BMC Genomics 14, 298.
Englert, M., Sheppard, K., Aslanian, A., Yates, J. R., and Soli, D. (2011). Archaeal 3′-phosphate RNA splicing ligase characterization identifies the missing component in tRNA maturation. Proc. Natl. Acad. Sci. U.S.A 108.
Fu, H., Feng, J., Liu, Q., Sun, F., Tie, Y., Zhu, J., Xing, R., Sun, Z., and Zheng, X. (2009). Stress induces tRNA cleavage by angiogenin in mammalian cells. FEBS Lett. 583, 437-442.
Gámbaro, F., Li Calzi, M., FagUndez, P., Costa, B., Greif, G., Mallick, E., Lyons, S., Ivanov, P., Witwer, K., Cayota, A., et al. (2020). Stable tRNA halves can be sorted into extracellular vesicles and delivered to recipient cells in a concentration-dependent manner. RNA Biol. 17, 1168-1182.
Garcia-Silva, M. R., Cura Das Neves, R. F., Cabrera-Cabrera, F., Sanguinetti, J., Medeiros, L. C., Robello, C., Naya, H., Fernandez-Calero, T., Souto-Padron, T., De Souza, W., et al. (2014). Extracellular vesicles shed by Trypanosoma cruzi are linked to small RNA pathways, life cycle regulation, and susceptibility to infection of mammalian cells. Parasitol. Res. 113, 285-304.
Geekiyanage, H., Rayatpisheh, S., Wohlschlegel, J. A., Brown, R., and Ambros, V. (2020). Extracellular microRNAs in human circulation are associated with miRISC complexes that are accessible to anti-AGO2 antibody and can bind target mimic oligonucleotides. Proc. Natl. Acad. Sci. U.S.A 117, 24213-24223.
Heitzer, E., Hague, I. S., Roberts, C. E. S., and Speicher, M. R. (2019). Current and future perspectives of liquid biopsies in genomics-driven oncology. Nat. Rev. Genet. 20, 71-88.
Ivanov, P., Emara, M. M., Villen, J., Gygi, S. P., and Anderson, P. (2011). Angiogenin-induced tRNA fragments inhibit translation initiation. Mol. Cell 43, 613-623.
Kaufmann, G. (2000). Anticodon nucleases. Trends Biochem. Sci. 25, 70-74.
Kim, H. K., Fuchs, G., Wang, S., Wei, W., Zhang, Y., Park, H., Roy-Chaudhuri, B., Li, P., Xu, J., Chu, K., et al. (2017). A transfer-RNA-derived small RNA regulates ribosome biogenesis. Nature 552, 57-62.
Kuscu, C., Kumar, P., Kiran, M., Su, Z., Malik, A., and Dutta, A. (2018). tRNA fragments (tRFs) guide Ago to regulate gene expression post-transcriptionally in a Dicer-independent manner. RNA 24, 1093-1105.
Lentini, J. M., Ramos, J., and Fu, D. (2018). Monitoring the 5-methoxycarbonylmethyl-2-thiouridine (mcm5s2U) modification in eukaryotic tRNAs via the γ-toxin endonuclease. RNA 24, 749-758.
Levin, A. A. (2019). Treating Disease at the RNA Level with Oligonucleotides. N. Engl. J. Med. 380, 57-70.
Li, G., Manning, A. C., Bagi, A., Yang, X., Gokulnath, P., Spanos, M., Howard, J., Chan, P. P., Sweeney, T., Kitchen, R., et al. (2022). Distinct Stress-Dependent Signatures of Cellular and Extracellular tRNA-Derived Small RNAs. Adv. Sci. 9.
Li, Y., Luo, J., Zhou, H., Liao, J. Y., Ma, L. M., Chen, Y. Q., and Qu, L. H. (2008). Stress-induced tRNA-derived RNAs: A novel class of small RNAs in the primitive eukaryote Giardia lamblia. Nucleic Acids Res. 36, 6048-6055.
Lyons, S. M., Fay, M. M., and Ivanov, P. (2018). The role of RNA modifications in the regulation of tRNA cleavage. FEBS Lett. 592, 2828-2844.
Lyons, S. M., Kharel, P., Akiyama, Y., Ojha, S., Dave, D., Tsvetkov, V., Merrick, W., Ivanov, P., and Anderson, P. (2021). eIF4G has intrinsic G-quadruplex binding activity that is required for tiRNA function. Nucleic Acids Res. 48, 6223-6233.
Mateescu, B., Kowal, E. J. K., van Balkom, B. W. M., Bartel, S., Bhattacharyya, S. N., Buzas, E. I., Buck, A. H., de Candia, P., Chow, F. W. N., Das, S., et al. (2017). Obstacles and opportunities in the functional analysis of extracellular vesicle RNA—An ISEV position paper. J. Extracell. Vesicles 6, 1286095.
Nechooshtan, G., Yunusov, D., Chang, K., and Gingeras, T. R. (2020). Processing by RNase 1 forms tRNA halves and distinct Y RNA fragments in the extracellular environment. Nucleic Acids Res. 48, 8035-8049.
Nolte'T Hoen, E. N. M., Buermans, H. P. J., Waasdorp, M., Stoorvogel, W., Wauben, M. H. M., and 'T Hoen, P. A. C. (2012). Deep sequencing of RNA from immune cell-derived vesicles uncovers the selective incorporation of small non-coding RNA biotypes with potential regulatory functions. Nucleic Acids Res. 40, 9272-9285.
O'Brien, K., Breyne, K., Ughetto, S., Laurent, L. C., and Breakefield, X. O. (2020). RNA delivery by extracellular vesicles in mammalian cells and its applications. Nat. Rev. Mol. Cell Biol. 21, 585-606.
Pawar, K., Shigematsu, M., Sharbati, S., and Kirino, Y. (2020). Infection-induced 5′-half molecules of tRNAHisGUG activate Toll-like receptor 7. PLoS Biol. 18, e3000982.
Popow, J., Englert, M., Weitzer, S., Schleiffer, A., Mierzwa, B., Mechtler, K., Trowitzsch, S., Will, C. L., Liihrmann, R., Soli, D., et al. (2011). HSPC117 is the essential subunit of a human tRNA splicing ligase complex. Science (80-.). 331, 760-764.
Qin, Y., Yao, J., Wu, D. C., Nottingham, R. M., Mohr, S., Hunicke-Smith, S., and Lambowitz, A. M. (2016). High-throughput sequencing of human plasma RNA by using thermostable group II intron reverse transcriptases. RNA 22, 111-128.
Sanadgol, N., König, L., Drino, A., Jovic, M., and Schaefer, M. R. (2022). Experimental paradigms revisited: oxidative stress-induced tRNA fragmentation does not correlate with stress granule formation but is associated with delayed cell death. Nucleic Acids Res. 50, 6919-6937.
Schwer, B., Sawaya, R., Ho, C. K., and Shuman, S. (2004). Portability and fidelity of RNA-repair systems. Proc. Natl. Acad. Sci. U.S.A 101, 2788-2793.
Skog, J., Wurdinger, T., van Rijn, S., Meijer, D. H., Gainche, L., Sena-Esteves, M., Curry, W. T. J., Carter, B. S., Krichevsky, A. M., and Breakefield, X. O. (2008). Glioblastoma microvesicles transport RNA and proteins that promote tumour growth and provide diagnostic biomarkers. Nat. Cell Biol. 10, 1470-1476.
Sorrentino, S. (2010). The eight human “canonical” ribonucleases: Molecular diversity, catalytic properties, and special biological actions of the enzyme proteins. FEBS Lett. 584, 2194-2200.
Srinivasan, S., Yeri, A., Cheah, P. S., Chung, A., Danielson, K., De Hoff, P., Filant, J., Laurent, C. D., Laurent, L. D., Magee, R., et al. (2019). Small RNA Sequencing across Diverse Biofluids Identifies Optimal Methods for exRNA Isolation. Cell 177, 446-462.
Tanaka, N., and Shuman, S. (2011). RtcB is the RNA ligase component of an Escherichia coli RNA repair operon. J. Biol. Chem. 286, 7727-7731.
Thomou, T., Mori, M. A., Dreyfuss, J. M., Konishi, M., Sakaguchi, M., Wolfrum, C., Rao, T. N., Winnay, J. N., Garcia-Martin, R., Grinspoon, S. K., et al. (2017). Adipose-derived circulating miRNAs regulate gene expression in other tissues. Nature 542, 450-455.
Thompson, D. M., Lu, C., Green, P. J., and Parker, R. (2008). tRNA cleavage is a conserved response to oxidative stress in eukaryotes. RNA 14, 2095-2103.
Tosar, J. P. (2021). Die hard: resilient RNAs in the blood. Nat. Rev. Mol. Cell Biol. 22, 373-373.
Tosar, J. P., and Cayota, A. (2020). Extracellular tRNAs and tRNA-derived fragments. RNA Biol. 17, 1149-1167.
Tosar, J. P., Gámbaro, F., Sanguinetti, J., Bonilla, B., Witwer, K. W., and Cayota, A. (2015). Assessment of small RNA sorting into different extracellular fractions revealed by high-throughput sequencing of breast cell lines. Nucleic Acids Res. 43, 5601-5616.
Tosar, J. P., Gambaro, F., Darre, L., Pantano, S., Westhof, E., and Cayota, A. (2018). Dimerization confers increased stability to nucleases in 5′ halves from glycine and glutamic acid tRNAs. Nucleic Acids Res. 46, 9081-9093.
Tosar, J. P., Segovia, M., Castellano, M., Gámbaro, F., Akiyama, Y., Fagúndez, P., Olivera, Á., Costa, B., Possi, T., Hill, M., et al. (2020). Fragmentation of extracellular ribosomes and tRNAs shapes the extracellular RNAome. Nucleic Acids Res. 48, 12874-12888.
Tosar, J. P., Witwer, K., and Cayota, A. (2021). Revisiting Extracellular RNA Release, Processing, and Function. Trends Biochem. Sci. 46, 438-445.
Tosar, J. P., Cayota, A., and Witwer, K. (2022). Exomeres and supermeres: Monolithic or diverse? J. Extracell. Biol. 1, e45.
Turchinovich, A., Weiz, L., Langheinz, A., and Burwinkel, B. (2011). Characterization of extracellular circulating microRNA. Nucleic Acids Res. 39, 7223-7233.
Valadi, H., Ekstrom, K., Bossios, A., Sjostrand, M., Lee, J. J., and Lotvall, J. O. (2007). Exosome-mediated transfer of mRNAs and microRNAs is a novel mechanism of genetic exchange between cells. Nat. Cell Biol. 9, 654-659.
Vickers, K. C., Palmisano, B. T., Shoucri, B. M., Shamburek, R. D., and Remaley, A. T. (2011). MicroRNAs are transported in plasma and delivered to recipient cells by high-density lipoproteins. Nat. Cell Biol. 13, 423-435.
Wei, Z., Batagov, A. O., Schinelli, S., Wang, J., Wang, Y., El Fatimy, R., Rabinovsky, R., Balaj, L., Chen, C. C., Hochberg, F., et al. (2017). Coding and noncoding landscape of extracellular RNA released by human glioma stem cells. Nat. Commun. 8, 1145.
Xiao, Y., Driedonks, T., Witwer, K. W., Wang, Q., and Yin, H. (2020). How does an RNA selfie work? EV-associated RNA in innate immunity as self or danger. J. Extracell. Vesicles 9, 1793515.
Yamasaki, S., Ivanov, P., Hu, G.-F., and Anderson, P. (2009). Angiogenin cleaves tRNA and promotes stress-induced translational repression. J. Cell Biol. 185, 35-42.
Zhang, Q., Jeppesen, D. K., Higginbotham, J. N., Graves-Deal, R., Trinh, V. Q., Ramirez, M. A., Sohn, Y., Neininger, A. C., Tanej a, N., McKinley, E. T., et al. (2021). Supermeres are functional extracellular nanoparticles replete with disease biomarkers and therapeutic targets. Nat. Cell Biol. 23, 1240-1254.
Zhang, Y., Zhang, Y., Shi, J., Zhang, H., Cao, Z., Gao, X., Ren, W., Ning, Y., Ning, L., Cao, Y., et al. (2014). Identification and characterization of an ancient class of small RNAs enriched in serum associating with active infection. J. Mol. Cell Biol. 6, 172-174.

1. A method of enzymatically repairing RNAs that are nicked or at least partially cleaved, comprising:
providing a biological sample containing RNAs that are nicked or at least partially cleaved;
purifying the nicked or at least partially cleaved RNAs contained in the biological sample, under a non-denaturing condition, to remove non-RNA components; and
treating the purified RNAs with at least one of the following:
(i) one or more enzymes that exhibit the activity of an RNA 3′ phosphatase or cyclic phosphatase and the activity of an RNA 5′ kinase, and an RNA ligase, or
(ii) a 3′-5′ RNA ligase,
thereby forming repaired RNAs from the nicked or at least partially cleaved RNAs.

2. The method of claim 1, wherein the nicked or at least partially cleaved RNAs comprise one or more RNAs selected from the group consisting of tRNA, rRNA, YRNA, 7SL RNA, 7SK RNA, snRNA, snoRNA, vaultRNA, Alu RNA, transposoable element-derived RNA, pri-microRNA, pre-microRNA, mRNA exons, mRNA introns, 5′ UTR, 3′ UTRs, and fragments and combinations thereof.

3. The method of claim 2, wherein the nicked or at least partially cleaved RNAs comprise tRNA, tRNA fragment (tRFs), tRNA-derived RNAs (tDRs), and/or tRNA half.

4. The method of claim 3, wherein at least a portion of the nicked or at least partially cleaved RNAs comprise single stranded tRNA half.

5. The method of claim 1, wherein the repaired RNA comprises a nucleotide sequence identical or substantially identical to the parent RNA from which the nicked or at least partially cleaved RNA was formed, in full-length or in substantial full-length.

6. The method of claim 1, wherein the treating is with (i)(a) a polynucleotide kinase (PNK) and an RNA ligase.

7. The method of claim 6, wherein the polynucleotide kinase is added together with, or followed by, the RNA ligase.

8. The method of claim 6 or 7, wherein the polynucleotide kinase is T4 polynucleotide kinase (PNK).

9. The method of claim 1, wherein the treating is with (i)(b) an RNA 3′ phosphatase or an RNA 2′,3′ cyclic phosphatase, an RNA 5′ kinase, and an RNA ligase.

10. The method of claim 9, wherein the RNA 3′ phosphatase or RNA 2′,3′ cyclic phosphatase, the RNA 5′ kinase, and the RNA ligase are added together, or in sequential order.

11. The method of claim 6, wherein the RNA ligase is T4 RNA ligase 1.

12. The method of claim 1, wherein the treating is with (ii) a 3′-5′ RNA ligase.

13. The method of claim 12, wherein the 3′-5′ RNA ligase is RtcB ligase.

14. The method of claim 6, wherein the treating is carried out at least in part in the presence of an adenosine triphosphate (ATP) or guanosine-5′-triphosphate (GTP).

15. The method of claim 1, wherein the non-denaturing condition comprises a silica-based solid phase extraction, a chromatographic method, RNA precipitation, or a combination thereof.

16. The method of claim 1, wherein the method is carried out in absence of a condition that results in denaturation of an RNA molecule.

17. The method of claim 16, wherein the condition comprises heating, an adapter ligation, a chemical denaturant, or combinations thereof.

18. The method of claim 16, wherein the condition comprises employing phenol.

19. The method of claim 1, wherein the source of the biological sample is a biofluid, a cell, a tissue, an organ, or any combination thereof.

20. The method of claim 1, wherein the biological sample is from an extracellular biofluid.

21. The method of claim 20, wherein the extracellular biofluid is blood, blood serum, blood plasma, urine, lymph, saliva, synovia, milk, cerebrospinal fluid, or a combination thereof.

22. The method of claim 1, wherein the biological sample is from a cell.

23. The method of claim 22, wherein the biological sample is from a cancerous cell.

24. The method of claim 22, wherein the nicked or at least partially cleaved RNAs are stress-induced tRNA halves.

25. The method of claim 1, wherein at least a portion of the nicked or partially cleaved RNAs are formed from nonvesicular extracellular RNAs.

26. The method of claim 1, wherein at least a portion of the nicked or partially cleaved RNAs are formed from extracellular RNAs.

27. The method of claim 5, wherein the repaired RNA comprises a nucleotide sequence identical or substantially identical to the parent RNA from which the nicked or at least partially cleaved RNA was formed, in full-length.

28. The method of claim 5, wherein the repaired RNA is 1-11 nucleotides shorter compared to its parent RNA from which the nicked or at least partially cleaved RNA was formed.

29. The method of claim 5, wherein the repaired RNA is 3-5 nucleotides shorter compared to its parent RNA from which the nicked or at least partially cleaved RNA was formed, losing a single-stranded overhang.

30. The method of claim 5, wherein the repaired RNA is 3-7 nucleotides shorter compared to its parent RNA from which the nicked or at least partially cleaved RNA was formed, losing an anticodon loop or a portion thereof.

31. A method for detecting RNAs from a biological sample, comprising:
providing a biological sample containing RNAs that are nicked or at least partially cleaved;
purifying the nicked or at least partially cleaved RNAs contained in the biological sample, under a non-denaturing condition, to remove non-RNA components;
treating the purified RNAs with at least one of the following:
(i) one or more enzymes that exhibit the activity of an RNA 3′ phosphatase or cyclic phosphatase and the activity of an RNA 5′ kinase, and an RNA ligase, or
(ii) a 3′-5′ RNA ligase, thereby repairing at least a portion of the nicked or partially cleaved RNAs; and
detecting the repaired RNAs.

32. The method of claim 31, wherein the detecting comprises sequencing, amplification, nucleic acid hybridization, or a combination thereof.

33. The method of claim 31, wherein the detecting comprises quantitative RT-PCR (RT-qPCR).

34. The method of claim 31, wherein the detecting comprises a form of high-throughput sequencing.

35. The method of claim 31, wherein the biological samples are treated with RNase inhibitors before or after the step of purifying the nicked or at least partially cleaved RNAs contained in the biological sample.

36. The method of claim 31, wherein the nicked or at least partially cleaved RNAs comprise one or more RNAs selected from the group consisting of tRNA, rRNA, YRNA, 7SL RNA, 7SK RNA, snRNA, snoRNA, vaultRNA, Alu RNA, transposable element-derived RNA, pri-microRNA, pre-microRNA, mRNA exons, mRNA introns, 5′ UTR, 3′ UTRs, and fragments and combinations thereof.

37. The method of claim 36, wherein the nicked or at least partially cleaved RNAs comprise tRNA, tRNA fragment (tRFs), tRNA-derived RNAs (tDRs), and/or tRNA half.

38. The method of claim 37, wherein at least a portion of the nicked or at least partially cleaved RNAs comprise single stranded tRNA half.

39. The method of claim 31, wherein the repaired RNA comprises a nucleotide sequence identical or substantially identical to the parent RNA from which the nicked or at least partially cleaved RNA was formed, in full-length or in substantial full-length.

40. The method of claim 31, wherein the treating is with (i)(a) a polynucleotide kinase (PNK) and an RNA ligase.

41. The method of claim 40, wherein the polynucleotide kinase is added together with, or followed by, the RNA ligase.

42. The method of claim 40 or 111, wherein the polynucleotide kinase is T4 polynucleotide kinase (PNK).

43. The method of claim 31, wherein the treating is with (i)(b) an RNA 3′ phosphatase or an RNA 2′,3′ cyclic phosphatase, an RNA 5′ kinase, and an RNA ligase.

44. The method of claim 43, wherein the RNA 3′ phosphatase or RNA 2′,3′ cyclic phosphatase, the RNA 5′ kinase, and the RNA ligase are added together, or in sequential order.

45. The method of claim 40, wherein the RNA ligase is T4 RNA ligase 1.

46. The method of claim 31, wherein the treating is with (ii) a 3′-5′ RNA ligase.

47. The method of claim 46, wherein the 3′-5′ RNA ligase is RtcB ligase.

48. The method of claim 40, wherein the treating is carried out at least in part in the presence of an adenosine triphosphate (ATP) or guanosine-5′-triphosphate (GTP).

49. The method of claim 31, wherein the non-denaturing condition comprises a silica-based solid phase extraction, a chromatographic method, RNA precipitation, or a combination thereof.

50. The method of claim 31, wherein the method is carried out in absence of a condition that results in denaturation of an RNA molecule.

51. The method of claim 50, wherein the condition comprises heating, a chemical denaturant, or combinations thereof.

52. The method of claim 50, wherein the condition comprises employing phenol.

53. The method of claim 31, wherein the source of the biological sample is a biofluid, a cell, a tissue, an organ, or any combination thereof.

54. The method of claim 31, wherein the biological sample is from an extracellular biofluid.

55. The method of claim 54, wherein the extracellular biofluid is blood, blood serum, blood plasma, urine, lymph, saliva, synovia, milk, cerebrospinal fluid, or a combination thereof.

56. The method of claim 31, wherein the biological sample is from a cell.

57. The method of claim 56, wherein the biological sample is from a cancerous cell.

58. The method of claim 56, wherein the nicked or at least partially cleaved RNAs are stress-induced tRNA halves.

59. The method of claim 31, wherein at least a portion of the nicked or partially cleaved RNAs are formed from nonvesicular extracellular RNAs.

60. The method of claim 31, wherein at least a portion of the nicked or partially cleaved RNAs are formed from extracellular RNAs.

61. The method of claim 39, wherein the repaired RNA comprises a nucleotide sequence identical or substantially identical to the parent RNA from which the nicked or at least partially cleaved RNA was formed, in full-length.

62. The method of claim 39, wherein the repaired RNA is 1-11 nucleotides shorter compared to its parent RNA from which the nicked or at least partially cleaved RNA was formed.

63. The method of claim 39, wherein the repaired RNA is 3-5 nucleotides shorter compared to its parent RNA from which the nicked or at least partially cleaved RNA was formed, losing a single-stranded overhang.

64. The method of claim 39, wherein the repaired RNA is 3-7 nucleotides shorter compared to its parent RNA from which the nicked or at least partially cleaved RNA was formed, losing an anticodon loop or a portion thereof.
